(19) United States
(12) Patent Application Publication
(10) Pub. No.: US 2019/0358341 A1
(43) Pub. Date: Nov. 28, 2019

(54) BIOCOMPATIBLE AND HYDROPHILIC POLYMER CONJUGATE FOR TARGETED DELIVERY OF AN AGENT

(71) Applicant: Commonwealth Scientific and Industrial Research Organisation, Acton, Australian Capital Territory (AU)

(72) Inventors: Timothy ADAMS, Parkville, Victoria (AU); John CHIEFARI, Acton, Australian Capital Territory (AU); Xiaojuan HAO, Acton, Australian Capital Territory (AU); Fei HUANG, Acton, Australian Capital Territory (AU); Laurence MEAGHER, Acton, Australian Capital Territory (AU); Judith SCOBLE, Acton, Australian Capital Territory (AU); Charlotte WILLIAMS, Parkville, Victoria (AU)

(21) Appl. No.: 16/472,333

(22) PCT Filed: Dec. 22, 2017
(86) PCT No.: PCT/AU2017/051448

§ 371 Date: Jun. 21, 2019

(30) Foreign Application Priority Data
Dec. 23, 2016 (AU) 2016905372

(51) Int. Cl. A61K 47/68; C08F 8/32; C08F 290/06; A61K 47/58

(52) U.S. Cl. CPC A61K 47/6883; C08F 8/32; A61K 47/6817; C08F 2810/40; A61K 47/6849; A61K 47/58; C08F 2438/03; C08F 290/062

(57) ABSTRACT

The present invention provides a biocompatible and hydrophilic polymer conjugate comprising a linear, aliphatic copolymer backbone to which is conjugated a binding moiety and an agent. The binding moiety is conjugated to an end of the copolymer backbone and facilitates targeted delivery of the agent. Also provided are methods for preparing such polymer conjugates via free radical polymerisation techniques such as reversible addition fragmentation chain transfer (RAFT) polymerisation and uses of such polymer conjugates in diagnosis or therapy.

### TECHNICAL FIELD

The present invention relates to biocompatible and hydrophilic polymer conjugates comprising a linear, aliphatic copolymer backbone to which is conjugated a binding moiety and an agent. The binding moiety is conjugated to an end of the copolymer backbone and facilitates targeted delivery of the agent. The invention also relates to methods for preparing such polymer conjugates via free radical polymerisation techniques such as reversible addition fragmentation chain transfer (RAFT) polymerisation and to uses of such polymer conjugates in diagnosis or therapy.

### BACKGROUND

Polymers have been used as carriers for a variety of agents, including drugs, diagnostic agents and imaging agents. A number of polymers of different chemical composition and architecture have been investigated as potential carriers.

One class of polymer described for the delivery of agents such as drugs are polymer-drug conjugates. These conjugates are generally composed of a polymer which is covalently linked to an agent, such as a therapeutic or diagnostic agent. The agent can be cleaved and released from the polymer in response to an appropriate stimulus.

Agents that are conjugated to polymers can have an increased circulation half-life. Additionally, the quantity of agent administered to a patient can be reduced when the agent is conjugated to a polymer. These benefits associated with polymer conjugated agents can contribute to an increase in the efficacy of the agent as well as a reduction in potential adverse side effects.

Polymers used in polymer-drug conjugates can be degradable or non-degradable when in a biological environment, with degradability influenced by the chemical structure and composition of the polymer chain. For example, degradable polymers can comprise monomer units coupled by degradable linkages such as ester, amide, anhydride, urethane or carbonate linkages, which form part of the polymer chain. Such degradable polymers can be synthesised by covalently reacting appropriately functionalised monomers, to couple the units of monomer through the degradable linkages. The linkages are susceptible to cleavage in vivo, leading to breakdown of the polymer chain and the formation of lower molecular weight fragments. In comparison, non-degradable polymers can have a polymer chain composed of monomeric units linked by carbon-carbon linkages. The carbon-carbon linkages can be formed through the polymerisation of unsaturated monomers and are not susceptible to breakdown in vivo.

While numerous polymer-drug conjugates have been described, there remains a need to provide polymer conjugates that can provide for improved delivery of an agent to target tissue.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

The present invention relates to biocompatible and hydrophilic polymer conjugates bearing a binding moiety and agent, which can provide for targeted delivery of the agent. Such polymer conjugates are also referred to herein as “polymer-agent conjugates” or “polymer conjugates”.

Broadly, the present invention relates to biocompatible, hydrophilic polymer conjugates comprising:
a linear, aliphatic copolymer backbone having two ends;
a binding moiety conjugated to an end of the copolymer backbone; and
at least one agent conjugated to the copolymer backbone.

The linear copolymer backbone of the polymer conjugate is derived from at least three different monomers.

It is one requirement of the invention that the linear copolymer backbone of the polymer conjugate is not a block copolymer.

In one aspect there is provided a biocompatible, hydrophilic polymer conjugate comprising:
a linear, aliphatic, statistical copolymer backbone having two ends and being derived from at least three different ethylenically unsaturated monomers;
a binding moiety conjugated to an end of the copolymer backbone; and
at least one agent conjugated to the copolymer backbone.

The polymer conjugates described herein can be suitable for the targeted delivery of an agent.

In the polymer conjugate, the agent is conjugated to the copolymer backbone at a position selected from an end of the backbone and pendant from the backbone, with the proviso that when the agent is conjugated at an end position then the agent and binding moiety are conjugated to different ends.

In a particular embodiment, the copolymer backbone is derived from at least three different ethylenically unsaturated monomers, wherein the different monomers each have different ethylenically unsaturated groups.

In one embodiment, the different monomers belong to classes of monomer selected from acrylic acid, methacrylic acid, acrylate, methacrylate, acrylamido, methacrylamido and vinyl ester.

In some embodiments, the copolymer backbone is a terpolymer. A skilled person would understand that a terpolymer is copolymer that is derived from three different ethylenically unsaturated monomers.

In one embodiment, terpolymers suitable as copolymer backbones in the polymer conjugates are derived from three different monomers, wherein each monomer has a different ethylenically unsaturated group.

The copolymer backbone of the polymer conjugate is preferably derived from hydrophilic ethylenically unsaturated monomers.

Polymer conjugates described herein comprise a binding moiety conjugated to an end of the linear copolymer backbone. In some embodiments, the binding moiety is a protein and may be selected from the group consisting of an antibody, an antibody fragment and an antigen binding fragment. In a particular embodiment, the binding moiety is a Fab′ fragment.

In another aspect there is provided a process for preparing a biocompatible, hydrophilic polymer conjugate, the process comprising the steps of:
(a) polymerising a monomer composition comprising at least three different ethylenically unsaturated monomers under conditions of free radical polymerisation to form a linear, aliphatic, statistical copolymer backbone having two ends, a first functional group for conjugating a binding moiety at a first end of the copolymer backbone, and a second functional group for conjugating an agent at a position selected from the second end of the copolymer backbone and pendant from the copolymer backbone;
(b) covalently reacting the first functional group with a binding moiety-containing molecule to conjugate the binding moiety to the first end of the copolymer backbone; and
(c) covalently reacting the second functional group with an agent-containing molecule to conjugate the agent to the copolymer backbone at a position selected from the second end of the copolymer backbone and pendant from the copolymer backbone.

In one embodiment, the monomer composition is polymerised under conditions of living free radical polymerisation, preferably reversible-addition-fragmentation-chain transfer (RAFT) polymerisation.

In a further aspect there is provided a process for preparing a biocompatible, hydrophilic polymer conjugate, the process comprising the steps of:
(a) polymerising a monomer composition comprising at least two different ethylenically unsaturated monomers and an ethylenically unsaturated monomer-agent conjugate under conditions of free radical polymerisation to thereby form a linear, aliphatic, statistical copolymer backbone having a pendant agent and a terminal functional group at one or both ends of the copolymer backbone;
(b) covalently reacting a binding moiety-containing molecule with a first terminal functional group at a first end of the copolymer backbone to conjugate the binding moiety to the first end; and
optionally (c) covalently reacting an agent-containing molecule with a second terminal functional group at a second end of the copolymer backbone to conjugate the agent to the second end.

In some embodiments of a process described herein, the monomer composition comprises a monomer-agent conjugate of formula (III):

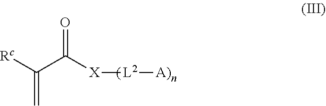

where:
   R^{c} is H or CH₃;
   X is selected from O or N;
   L² represents a linking moiety;
   A represents an agent; and
   n represents the number of (-L²-A) groups attached to X and is 1 or 2.

In yet a further aspect there is provided a method of alleviating, treating or preventing a disease or disorder in a subject comprising the step of administering to the subject, an effect amount of a polymer conjugate of any one of the embodiments described herein.

In yet a further aspect there is provided a method of delivering an agent to a target cellular or tissue site in a subject, the method comprising the step of administering an effective amount of a polymer conjugate of any one of the embodiments described herein to the subject.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### BRIEF DESCRIPTION OF THE FIGURES

Embodiments of the invention will now be described with reference to the following non-limiting figures in which:

FIG. 1 shows graphs illustrating Europium-ligand competition assays comparing the ability of 528 Fab′-polymer conjugates to compete for binding to soluble EGFR in the presence of Eu-EGF;

FIG. 2 shows a graph illustrating dose-response inhibition of EGFR tyrosine phosphorylation in ACHN carcinoma cells by a 528 Fab′-polymer conjugate having 10 kDa PEG (comparative) and a 528 Fab′-polymer conjugate having 10 kDa p(HPMA) RAFT polymer;

FIG. 3 shows a graph illustrating changes in plasma concentration as a function of time for different aliphatic polymers tested after IV administration of 5 mg/kg polymer to rats;

FIG. 4 shows graphs illustrating the clearance rate of different aliphatic polymers tested as a function of (A) the molecular weight of the polymers or (B) their gel filtration elution volume;

FIG. 5 shows a graph illustrating pharmacokinetic profiles for various Fab′-linked polymers, with each data point being the average for three rats; and

FIG. 6 shows a graph illustrating the efficacy of various Fab′-polymer-drug conjugates (APDC) in relation to human epidermoid carcinoma volume (mm³) derived from A431 cells grown in athymic nude scid mice, over 42 days.

### DETAILED DESCRIPTION

As used herein, the singular forms “a”, “an”, and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein the terms “treating” and “treatment” refer to any and all uses which remedy a condition or symptom, or otherwise hinder, retard, suppress or reverse the progression of a condition or disease or other undesirable symptoms in any way whatsoever. Thus, the terms “treating” and “treatment” and the like are to be considered in their broadest context.

For example, treatment does not necessarily imply that a patient is treated until total recovery. In the context of the present disclosure “treatment” may involve reducing or ameliorating the occurrence of a symptom or highly undesirable event associated with the disorder or an irreversible outcome of the progression of the disorder but may not of itself prevent the initial occurrence of the event or outcome. Accordingly, treatment includes the amelioration of one or more symptoms of a particular disorder or preventing or otherwise reducing the risk of developing a particular disorder.

The present invention broadly relates to biocompatible and hydrophilic polymer conjugates comprising a binding moiety and an agent, which is useful for the targeted delivery of the agent to a localised site.

The present invention broadly relates to a biocompatible, hydrophilic polymer conjugate comprising:
a linear, aliphatic copolymer backbone having two ends;
a binding moiety conjugated to one end of the copolymer backbone; and
at least one agent conjugated to the copolymer backbone.

It is a proviso that in the polymer conjugates described herein that the linear, aliphatic copolymer backbone is not a block copolymer. That is, the copolymer backbone is not one having separate and discrete blocks of different composition, where each discrete block is composed of different polymerised monomers.

Suitably, the linear aliphatic polymer backbone is a statistical copolymer derived from at least three co-monomers.

In a first aspect the present invention provides a biocompatible, hydrophilic polymer conjugate comprising:
a linear, aliphatic, statistical copolymer backbone having two ends and being derived from at least three different ethylenically unsaturated monomers;
a binding moiety conjugated to an end of the copolymer backbone; and
at least one agent conjugated to the copolymer backbone.

The polymer conjugate of the present invention is biocompatible and hydrophilic and is amenable for use in biomedical applications where the targeted delivery of an agent is desired.

By “biocompatible” is meant that the polymer conjugate is minimally toxic or non-toxic to a biological environment, such as living tissue or a living organism.

By “hydrophilic” is meant that the polymer conjugate has an affinity for water and is thus compatible with an aqueous solvent and may be soluble in an aqueous solvent. Preferably, the polymer conjugate is soluble in water. In some embodiments, the polymer conjugate may have a solubility in water of at least 10 g of polymer per 100 g of water at 25° C.

A “polymer conjugate” of the invention is a covalent conjugate of a copolymer, at least one binding moiety and at least one agent. The agent may be a therapeutic agent, a diagnostic agent, or research reagent.

The polymer conjugates of the invention preferably do not self-assemble or associate into structured assemblies, e.g. micelles.

Polymer conjugates of the invention comprise a statistical copolymer backbone. In such embodiments, the copolymer backbone is a linear aliphatic molecule composed of statistically distributed polymerised residues derived from at least three different ethylenically unsaturated co-monomers. A skilled person would understand that the different co-monomers become incorporated into the structure of the linear polymer chain due to chain addition of the co-monomers as polymerisation proceeds. The incorporated monomers form polymerised residues in the resulting copolymer. Polymerised residues may be regarded as monomeric units of the copolymer.

A skilled person would understand that a “statistical copolymer” is a macromolecule in which the sequential distribution of the monomeric units obeys known statistical laws. An example of a statistical copolymer is a macromolecule in which the sequential distribution of monomeric units follows Markovian statistics.

Statistical copolymers are formed when the different co-monomers are copolymerised simultaneously under free radical polymerisation conditions. Under such conditions, the ethylenically unsaturated moieties of the co-monomers react to link the co-monomers together via covalent carbon-carbon bonds. The incorporation and distribution of co-monomers in the statistical copolymer can therefore be dictated by the relative reactivity (i.e. reactivity ratio) of the different co-monomers. Thus co-monomer reactivity can influence the composition of the copolymer.

Ethylenically unsaturated co-monomers described herein may be selected from those having reactivity ratios that facilitate formation of a statistical copolymer.

In some embodiments, statistical copolymers may have a random distribution of monomeric units derived from the different co-monomers.

Statistical copolymers described herein are distinguished from block copolymers as block copolymers often require monomer addition and polymerisation to be controlled to achieve a predetermined and controlled distribution of monomeric units in the copolymer, which thus generate the block composition.

The copolymer backbone is a linear molecule and has two ends. The two ends are terminal, opposing ends and may be referred to herein as the alpha (c) and omega (co) ends of the copolymer. The two ends of the copolymer may also be referred to herein as a first end and a second end of the copolymer, to denote that they are different ends of the linear molecule.

The copolymer backbone of the polymer conjugate is also an aliphatic molecule. By “aliphatic” is meant that the copolymer backbone is a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. The copolymer backbone is thus formed of carbon atoms that are linked together via carbon-carbon bonds. The chain of carbon atoms forming the copolymer backbone is in general not interrupted by heteroatoms, such as oxygen, nitrogen or sulfur atoms. In one embodiment, the copolymer backbone is a straight-chain hydrocarbon moiety.

A linear, aliphatic copolymer backbone would thus be understood by one skilled in the art to be a macromolecule composed of monomeric units that are linked via carbon-carbon bonds along its linear axis. The length of the linear copolymer chain would be dictated by the number of monomeric units incorporated in the copolymer.

The copolymer backbone of the polymer conjugate is formed through the polymerisation of at least three different ethylenically unsaturated co-monomers under free radical polymerisation conditions. The copolymer backbone thus contains polymerised residues derived from the different co-monomers.

In some embodiments, the copolymer is a terpolymer that is formed through the polymerisation of three different ethylenically unsaturated co-monomers.

In some other embodiments the copolymer may be formed through the polymerisation of more than three different ethylenically unsaturated co-monomers.

In one set of embodiments, the linear copolymer backbone comprises statistically distributed polymerised residues of at least three different ethylenically unsaturated hydrophilic monomers. The hydrophilic monomers can assist to confer hydrophilic properties to the polymer conjugate.

Ethylenically unsaturated groups as described herein comprise an ethylenically unsaturated moiety. Ethylenically unsaturated moieties may be carbon-carbon double bonds or carbon-carbon triple bonds. The ethylenically unsaturated moiety may be a part of a ring structure or a terminal group.

Ethylenically unsaturated monomers as described herein comprise at least one ethylenically unsaturated group, which is polymerisable under free radical polymerisation conditions. In one preference, the monomers each contain a single polymerisable ethylenically unsaturated group. The presence of a single polymerisable ethylenically unsaturated group can help minimise the occurrence of crosslinking reactions and thus help ensure that the polymerisation reaction generates a linear copolymer.

Ethylenically unsaturated monomers having a single polymerisable ethylenically unsaturated group may also be regarded as mono-substituted monomers.

Ethylenically unsaturated co-monomers may be considered to be different from one another by having different chemical environments surrounding the ethylenically unsaturated moiety of the monomers.

For instance, there may be different chemical substituent groups directly covalently bonded to the carbon atoms of the ethylenically unsaturated moiety of the different co-monomers. Different substituent groups bonded to the ethylenically unsaturated moieties can thus produce ethylenically unsaturated groups that are not identical in chemical structure. Accordingly, such co-monomers will generally be considered to be different from one another.

A range of suitable ethylenically unsaturated monomers would be known to a skilled person. Preferred ethylenically unsaturated monomers may be vinyl, acryloyl or methacryloyl monomers.

Examples of acryloyl and methacryloyl monomers include acrylic acid, methacrylic acid, acrylate, methacrylate, acrylamido and methacrylamido monomers.

In one embodiment, the polymer conjugate of the invention comprises a linear copolymer derived from at least three different ethylenically unsaturated co-monomers, wherein the co-monomers are selected from acrylic acid, methacrylic acid, acrylate, methacrylate, acrylamido, methacrylamido and vinyl ester monomers.

The acrylic acid, methacrylic acid, acrylate, methacrylate, acrylamido, methacrylamido and vinyl ester groups are each considered to be different polymerisable ethylenically unsaturated groups.

Monomers containing acrylic acid, methacrylic acid, acrylate, methacrylate, acrylamido, methacrylamido and vinyl ester groups can be categorised into different classes, which are defined by reference to the different chemical structures of the ethylenically unsaturated groups, resulting in different types of polymerisable groups.

A skilled person would understand that acrylic acid, methacrylic acid, acrylate monomers, methacrylate monomers, acrylamido monomers and methacrylamido monomers would each have a carbonyl (—C═O) functionality directly covalently bonded to the ethylenically unsaturated moiety of the monomer, which is a carbon-carbon double bond.

However, the above acryloyl and methacryloyl monomers differ from one another in that acrylate and methacrylate monomers are esters and have an oxygen atom containing substituent group (—OR) covalently bonded to the carbonyl. In comparison, acrylamido and methacrylamido monomers have a nitrogen atom containing substituent group (—NR) covalently bonded to the carbonyl to form an amide. Acrylic acid and methacrylic acid monomers are carboxylic acids and have a hydroxyl moiety (—OH) covalently bonded to the carbonyl.

Acrylic acid, acrylate and acrylamide monomers also differ from methacrylic acid, methacrylate and methacrylamido monomers in that the three latter monomer classes have a methyl substituent directly covalently bonded to the carbon-carbon double bond, at the carbon atom that is alpha to the carbonyl. In acrylic acid, acrylates and acrylamides, the methyl substituent is absent.

Acrylate, methacrylate, acrylamide and methacrylamido monomers may have one or more substituent groups (i.e. R groups) bonded to either the oxygen atom of the ester moiety or the nitrogen atom of the amido moiety of these monomers. The substituent group or groups can provide functionalities pendant from the copolymer backbone. A skilled person would understand that such substituent groups are not directly covalently bonded to the ethylenically unsaturated moiety (e.g. a carbon-carbon double bond) of the monomers, but may be spatially separated from the unsaturated moiety by one or more atoms (e.g. oxygen, carbon or nitrogen atoms).

Monomers belonging to the class of acrylate monomers include but are not limited to acryloyl esters such as 2-hydroxyethyl acrylate, 2-methoxyethyl acrylate, 2-(diethylene glycol) ethyl acrylate, poly(ethylene glycol) acrylate, poly(ethylene glycol) methyl ether acrylate, 2-(diethylamino) ethyl acrylate, 3-(dimethylamino) propyl acrylate, N-acryloxysuccinimide, 3-[[2-(acryloyloxy)ethyl]dimethylammonio]propionate, 2-acryloyloxyethyl phosphorylcholine, and [2-(acryloyloxy)ethyl]dimethyl-(3-sulfopropyl) ammonium hydroxide.

Monomers belonging to the class of methacrylate monomers include but are not limited to methacryloyl esters such as poly(ethylene glycol) methacrylate, poly(ethylene glycol) methyl ether methacrylate, di(ethylene glycol) methyl ether methacrylate, 2 hydroxyethyl methacrylate, 2-aminoethyl methacrylate hydrochloride, 3-sulfopropyl methacrylate potassium salt, 3-[[2-(methacryloyloxy)ethyl]dimethylammonio]propionate, 2-methacryloyloxyethyl phosphorylcholine, and [2-(methacryloyloxy)ethyl]dimethyl-(3-sulfopropyl) ammonium hydroxide.

Monomers belonging to the class of acrylamido monomers include but are not limited to unsubstituted, N-monosubstituted and N,N-disubstituted acryloyl amides such as N-(2-hydroxypropyl) acrylamide, N-acryloylamido-ethoxyethanol, N,N-dimethylacrylamide, N,N-diethylacrylamide, N-(2-hydroxyethyl) acrylamide, N [Tris(hydroxymethyl)methyl]acrylamide, acrylamide, N-acryloylmorpholine, N propyl acrylamide, N isopropyl acrylamide, N-(2-propynyl)-acrylamide, N-(3-azidopropyl) acrylamide, (3 acrylamidopropyl) trimethylammonium chloride, N-carboxyethyl acrylamide, and 2-acrylamido-2-methyl-1-propane sodium sulfonate.

Monomers belonging to the class of methacrylamido monomers include but are not limited to unsubstituted, N-monosubstituted and N,N-disubstituted methacryloyl amides such as N-(2-hydroxypropyl) methacrylamide, N-(2 hydroxyethyl) methacrylamide, methacrylamide, [3 (methacryloylamino)propyl]trimethylammonium chloride, and N-(3-azidopropyl) methacrylamide.

Vinyl ester monomers are another class of ethylenically unsaturated monomer. Vinyl monomers generally contain an unsaturated moiety which is a carbon-carbon double bond, with a substituent covalently bonded to the carbon-carbon double bond. In the case of vinyl esters, an oxygen atom is directly bonded to the carbon-carbon double bond, with a carbonyl subsequently bonded to the oxygen atom.

Monomers belonging to the class of vinyl esters may have a range of substituent groups (R groups) bonded to the carbonyl of the ester. One example of a vinyl ester is vinyl acetate. The ester may be hydrolysed after formation of the copolymer backbone to generate a hydroxy group, which is pendant from the copolymer.

In one form, the polymer conjugate may comprise a copolymer derived from at least three different ethylenically unsaturated monomers that belong to the same class of monomer yet which differ from one another with respect to the substituent linked to the ethylenically unsaturated group of the monomer. As an example, the copolymer may be derived from at least three acrylamido monomers that each have the same type of ethylenically unsaturated group yet have a different type of substituent group (i.e. R group) bonded to the nitrogen atom of the acrylamido moiety of the monomers. This may be illustrated by reference to the model compound shown below, where co-monomers belonging to the same class may have identical groups A, B, C and D directly bonded to the unsaturated moiety, but different R substituent groups. Since groups A, B, C and D are identical, the co-monomers would thus have the same type of ethylenically unsaturated group and belong to the same monomer class.

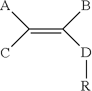

In another form, the polymer conjugate may comprise a copolymer derived from at least three different ethylenically unsaturated monomers, where the different monomers each belong to a different class. Accordingly, in such embodiments, the copolymer is derived from at least three different classes of ethylenically unsaturated monomer. Monomers belonging to different classes differ with respect to one another in relation to the type of ethylenically unsaturated group in the monomers. This may be illustrated by reference to the model compound shown below, where co-monomers belonging to different classes have one or more different substituents directly covalently bonded to the ethylenically unsaturated moiety. That is, at least one of groups A, B, C and D, which are directly bonded to the unsaturated moiety, differ between the different types of co-monomers, to thereby provide different ethylenically unsaturated groups.

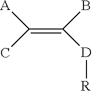

In some embodiments, the copolymer may be derived from a first monomer, a second monomer and a third monomer, wherein the first, second and third monomers differ in respect of the ethylenically unsaturated group and thus belong to different classes of monomer as described herein.

In addition to differing with respect to the ethylenically unsaturated group, co-monomers belonging to different classes may also differ with respect to the substituent group (i.e. R group) covalently linked to the unsaturated group of the monomers.

In one embodiment, the polymer conjugate of the invention comprises a copolymer backbone derived from at least three different ethylenically unsaturated hydrophilic monomers. Copolymer backbones derived from hydrophilic monomers can help to confer hydrophilicity to the polymer conjugate.

The term “hydrophilic” as used in relation to a monomer means that the monomer has an affinity for water and is at least compatible with an aqueous solvent. Preferably, the monomer is soluble in an aqueous solvent, such as water or a solvent mixture comprising water (e.g. a mixture of water and a water-miscible organic solvent). In some embodiments, a hydrophilic monomer may have solubility in water of at least 10 g of monomer per 100 g of water at 25° C.

However, it is contemplated that the linear copolymer backbone of the polymer conjugate may be derived from monomers that are not considered hydrophilic. However, provided that these monomers do not adversely affect the desired overall hydrophilicity of the polymer conjugate per se, then such monomers can be used.

In some instances, if desired, polymerised resides in the copolymer that are derived from non-hydrophilic (i.e. hydrophobic) monomers can be modified by a range of chemical processes to convert them into hydrophilic residues. For examples, pendant substituent groups (R groups) in polymerised residues derived from hydrophobic monomers may be modified though hydrolysis or substitution reactions to convert them into hydrophilic moieties.

In some embodiments, the linear copolymer backbone comprises statistically distributed polymerised residues of at least three different ethylenically unsaturated hydrophilic monomers.

In a particular embodiment, the copolymer backbone of the polymer conjugate is a linear, aliphatic terpolymer having statistically distributed polymerised residues of three different ethylenically unsaturated hydrophilic co-monomers. Preferably, the different hydrophilic co-monomers each have a different type of ethylenically unsaturated group.

In one preference, the copolymer backbone comprises polymerised residues derived from at least three different ethylenically unsaturated hydrophilic monomers belonging to classes of monomer selected from acrylic acid, methacrylic acid, acrylate, methacrylate, acrylamido, methacrylamido and vinyl ester, wherein each different monomer belongs to a different class. Hydrophilic monomers belonging to these classes may be selected from those listed above.

The linear copolymer backbone of the polymer conjugate of the invention may, and preferably will, comprise one or more functional groups.

In one preference, the linear copolymer backbone comprises one or more pendant functional groups. Such functional groups are pendant from the main chain of the linear copolymer backbone. By being “pendant”, the functional group does not directly form part of the chain of carbon atoms forming the copolymer backbone.

Pendant functional groups may be capable of participating in hydrogen bonding interactions with water and in this way, help to promote the hydrophilicity of the copolymer backbone and hence the polymer conjugate.

Pendant functional groups may also be capable of participating in covalent reactions that facilitate conjugation and attachment of an agent, such as a therapeutic agent, diagnostic agent or research agent, to the copolymer backbone to thereby form the polymer conjugate.

The pendant functional group may be introduced when an ethylenically unsaturated monomer having a substituent group (i.e. “R” group) comprising a functional group forms a monomeric unit of the copolymer backbone. The copolymer backbone therefore comprises a polymerised residue of the monomer, with the functional group remaining pendant from the backbone. Exemplary functional groups may be hydroxyl, amino, carboxyl, carbonyl, sulfate, sulfonate, phosphate and succinimido, preferably hydroxyl, succinimido, alkynyl, azido, and combinations thereof.

Substituent groups containing zwitterionic functional groups, such as carboxybetaine, sulphobetaine and phosphobetaine groups, are also contemplated in some embodiments.

Zwitterionic functional groups comprise a moiety having both positive and negative charge. Some examples of zwitterionic functional groups are illustrated below:

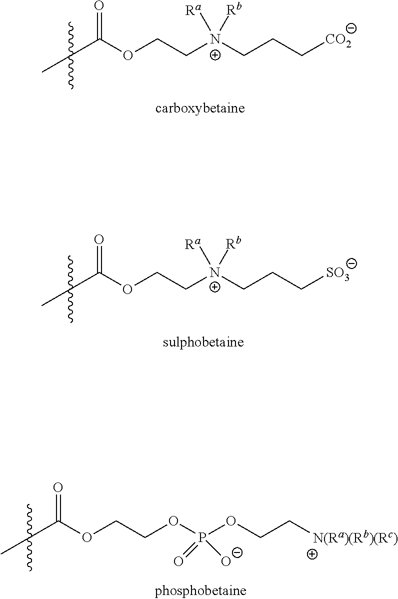

where R^{a}, R^{b}, R^{c} are each independently selected from hydrogen and C1-C6 alkyl (preferably C1-C2 alkyl, more preferably methyl).

In some other embodiments, the linear aliphatic copolymer backbone of the polymer conjugate does not comprise a polymerised residue derived from an ethylenically unsaturated zwitterionic monomer. Thus in some embodiments it is a proviso that the monomers used in formation of the linear copolymer backbone are not zwitterionic, such that the resulting copolymer does not comprise a pendant zwitterionic group.

In one form, the copolymer backbone is derived from at least three different ethylenically unsaturated hydrophilic monomers, the different monomers being selected from the group consisting of N-(2-hydroxypropyl) methacrylamide, N-(2-hydroxypropyl) acrylamide, 2-hydroxyethyl acrylate, 2-methoxyethyl acrylate, 2-(diethylene glycol) ethyl acrylate, poly(ethylene glycol) acrylate, poly(ethylene glycol) methacrylate, poly(ethylene glycol) methyl ether acrylate, poly(ethylene glycol) methyl ether methacrylate, N-acryloylamido-ethoxyethanol, N,N-dimethylacrylamide, N,N-diethylacrylamide, N-(2-hydroxyethyl) acrylamide, N-(2-hydroxyethyl) methacrylamide, N-[Tris(hydroxymethyl)methyl]acrylamide, acrylamide, N-acryloylmorpholine, N-propyl acrylamide, N-isopropyl acrylamide, methacrylamide, di(ethylene glycol) methyl ether methacrylate, 2-hydroxyethyl methacrylate, 2-(dimethylamino) ethyl acrylate, 2-(diethylamino) ethyl acrylate, 3-(dimethylamino) propyl acrylate, N-acryloyloxysuccinimide, (3-acrylamidopropyl) trimethylammonium chloride, 2-aminoethyl methacrylate hydrochloride, [3-(methacryloylamino)propyl]trimethylammonium chloride, 2-carboxyethyl acrylate, acrylic acid, N-carboxyethyl acrylamide, 2-acrylamido-2-methyl-1-propane sodium sulfonate, 3-sulfopropyl methacrylate potassium salt, methacrylic acid, N-acryloxysuccinimide, 3-[[2-(methacryloyloxy)ethyl]dimethylammonio]propionate, 2-methacryloyloxyethyl phosphorylcholine, [2-(methacryloyloxy)ethyl]dimethyl-(3-sulfopropyl) ammonium hydroxide, 3-[[2-(acryloyloxy)ethyl]dimethyl-ammonio]propionate, 2-acryloyloxyethyl phosphorylcholine, [2-(acryloyloxy)ethyl]dimethyl-(3-sulfopropyl) ammonium hydroxide, N-(2-propynyl)-acrylamide, N-(3-azidopropyl)-acrylamide, N-(3-azidopropyl)-methacrylamide and vinyl acetate.

In some embodiments it can be desirable that polymerised monomer residues in the copolymer backbone are neutral and carry no net charge at physiological pH (approximately pH 7.4). This can help ensure that the polymer conjugate carries no net charge at physiological pH. This can be desirable as charged polymer conjugates can induce adverse effects in the physiological environment. For example, cationic resides can induce cytotoxicity.

In one form, a linear aliphatic copolymer backbone that comprises statistically distributed polymerised residues derived from at least three different ethylenically unsaturated monomers can have a general structure represented by formula (Ia):

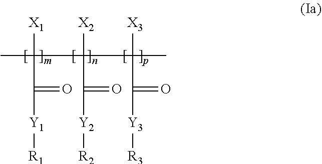

where:
X₁, X₂ and X₃ may be the same or different and are each independently selected from H and CH₃;
Y₁, Y₂ and Y₃ may be the same or different and are each independently selected from O and NR, where R is H or C1-C6 alkyl (preferably C1-C4 alkyl, most preferably methyl);
R₁, R₂ and R₃ may be the same or different and are each substituent groups; and
m, n and p represent the number of repeat units for a polymerised residue and are each an integer of at least 1,
with the proviso that:
(i) when X₁ is H, X₂ is CH₃, and X₃ is H, then Y₁ and Y₃ are different,
(ii) when X₁ is H, X₂ is H, and X₃ is CH₃, then Y₁ and Y₂ are different,
(iii) when X₁ is CH₃, X₂ is CH₃, and X₃ is H, then Y₁ and Y₂ are different,
(iv) when X₁ is CH₃, X₂ is H, and X₃ is CH₃, then Y₁ and Y₃ are different, and
(v) when X₁, X₂ and X₃ are the same and Y₁, Y₂ and Y₃ are the same, then R₁, R₂ and R₃ are each different.

The substituent groups R₁, R₂ and R₃ in formula (Ia) may in some embodiments be linear or cyclic alkyl or linear or cyclic heteroalkyl. Linear alkyl or heteroalkyl may be branched or unbranched. Cyclic alkyl or heteroalkyl can comprise from 6 to 8 ring atoms.

One or more of the substituent groups R₁, R₂ and R₃ may also comprise a functional group.

The functional group may be selected from hydroxyl, amino, amido, carboxyl, carbonyl, sulfate, sulfonate, phosphate, succinimido, alkynyl, azido, and combinations thereof. In one embodiment, the substituent groups R₁, R₂ and R₃ each independently comprise a functional group selected from hydroxyl, succinimido, carboxybetaine, sulphobetaine and phosphobetaine.

In one preference, the copolymer backbone comprises polymerised monomer residues derived from at least three different ethylenically unsaturated hydrophilic monomers, wherein the different monomers are selected from the group consisting of N-(2-hydroxypropyl) methacrylamide, N-(2-hydroxypropyl) acrylamide, poly(ethylene glycol) acrylate, poly(ethylene glycol) methacrylate, N-acryloylmorpholine, N-isopropyl acrylamide, and N-acryloxysuccinimide.

In one embodiment, at least one of the polymerised monomer residues in the linear copolymer backbone comprises an agent, such as a therapeutic or diagnostic agent, conjugated thereto.

In some embodiments, at least one of the polymerised monomers forming a monomeric unit of the copolymer backbone comprises a functional group that is capable of covalently reacting with an agent-containing molecule, to facilitate conjugation of the agent to the copolymer backbone. Following the covalent reaction, the result is a copolymer backbone comprising a monomeric unit comprising an agent conjugated thereto.

In one embodiment, the linear copolymer backbone of the polymer conjugate comprises polymerised residues derived from:
(a) a first co-monomer selected from N-(2-hydroxypropyl)methacrylamide and N-(2-hydroxypropyl) acrylamide;
(b) a second co-monomer selected from 2-hydroxyethyl acrylate, 2-methoxyethyl acrylate, 2-(diethylene glycol) ethyl acrylate, poly(ethylene glycol) acrylate, poly(ethylene glycol) methacrylate, poly(ethylene glycol) methyl ether acrylate, poly(ethylene glycol) methyl ether methacrylate, N-acryloylamido-ethoxyethanol, N,N-dimethylacrylamide, N,N-diethylacrylamide, N-(2-hydroxyethyl) acrylamide, N-(2-hydroxyethyl) methacrylamide, N-[Tris(hydroxymethyl)methyl]acrylamide, acrylamide, N-acryloylmorpholine, N-propyl acrylamide, N-isopropyl acrylamide, methacrylamide, di(ethylene glycol) methyl ether methacrylate, 2-hydroxyethyl methacrylate, 2-(dimethylamino) ethyl acrylate, 2-(diethylamino) ethyl acrylate, 3-(dimethylamino) propyl acrylate, (3-acrylamidopropyl) trimethylammonium chloride, 2-aminoethyl methacrylate hydrochloride, [3-(methacryloylamino)propyl]trimethylammonium chloride, 2-carboxyethyl acrylate, acrylic acid, N-carboxyethyl acrylamide, 2-acrylamido-2-methyl-1-propane sodium sulfonate, 3-sulfopropyl methacrylate potassium salt, methacrylic acid, 3-[[2-(methacryloyloxy)ethyl]dimethylammonio]propionate, 2-methacryloyloxyethyl phosphorylcholine, [2-(methacryloyloxy)ethyl]dimethyl-(3-sulfopropyl) ammonium hydroxide, 3-[[2-(acryloyloxy)ethyl]dimethylammonio]propionate, 2-acryloyloxyethyl phosphorylcholine, [2-(acryloyloxy)ethyl]dimethyl-(3-sulfopropyl) ammonium hydroxide, N-(2-propynyl)-acrylamide, N-(3-azidopropyl)-acrylamide, N-(3-azidopropyl)-methacrylamide, and vinyl acetate; and
(c) a third co-monomer selected from an acryloyl or methacryloyl monomer comprising a functional group capable of reacting with an agent-containing molecule, and an acryloyl or methacryloyl monomer comprising an agent conjugated thereto.

It is a proviso that the first, second and third co-monomers described above are different ethylenically unsaturated monomers. Preferably, the first, second and third co-monomers belong to different classes of ethylenically unsaturated monomer. Examples of different classes of ethylenically unsaturated monomer are described herein.

In one embodiment, the third-co-monomer is an acryloyl monomer comprising a functional group capable of reacting with an agent-containing molecule. An example of such a functionalised acryloyl monomer is N-acryloxysuccinimide. A skilled person would understand that the succinimido functional group may react with an appropriately functionalised agent-containing molecule to enable the agent (e.g. a therapeutic agent) to be conjugated to the copolymer backbone through a polymerised residue derived from the N-acryloxysuccinimide monomer. In this manner, functionalisation of the copolymer backbone post-polymerisation can facilitate loading of the agent and formation of the polymer conjugate.

In an alternative embodiment, the third co-monomer is a monomer-agent conjugate of formula (I) or (II) as described herein. In such embodiments, the agent becomes incorporated into the polymer conjugate as a result of the monomer-agent conjugate being polymerised with the first and second monomers.

The first, second and third co-monomers may be present in the copolymer backbone in a suitable ratio.

In one embodiment, the molar ratio between the first and second co-monomers in the copolymer backbone may be in the range of from 4:1 to 1:4, preferably a molar ratio in the range of from about 2:1 to 1:1.

In some embodiments, the first and second co-monomers may together form at least 65%, at least 70%, at least 80% or at least 90% of polymerised residues in the copolymer backbone, on a molar basis.

The third co-monomer may be present in a desired amount. In some embodiments, the third co-monomer is present in an amount of from about 5 to 30 mol % of the copolymer backbone, preferably from about 10 to 20 mol % of the copolymer backbone.

In one set of embodiments, the linear copolymer backbone comprises polymerised residues derived from:
a first co-monomer which is N-(2-hydroxypropyl)methacrylamide;
a second co-monomer selected from 2-hydroxyethyl acrylate, 2-methoxyethyl acrylate, 2-(diethylene glycol) ethyl acrylate, poly(ethylene glycol) acrylate, poly(ethylene glycol) methacrylate, poly(ethylene glycol) methyl ether acrylate, poly(ethylene glycol) methyl ether methacrylate, N-acryloylamido-ethoxyethanol, N,N-dimethylacrylamide, N,N-diethylacrylamide, N-(2-hydroxyethyl) acrylamide, N-(2-hydroxyethyl) methacrylamide, N-[Tris(hydroxymethyl)methyl]acrylamide, acrylamide, N-acryloylmorpholine, N-propyl acrylamide, N-isopropyl acrylamide, methacrylamide, di(ethylene glycol) methyl ether methacrylate, 2-hydroxyethyl methacrylate, 2-(dimethylamino) ethyl acrylate, 2-(diethylamino) ethyl acrylate, 3-(dimethylamino) propyl acrylate, (3-acrylamidopropyl) trimethylammonium chloride, 2-aminoethyl methacrylate hydrochloride, [3-(methacryloylamino)propyl]trimethylammonium chloride, 2-carboxyethyl acrylate, acrylic acid, N-carboxyethyl acrylamide, 2-acrylamido-2-methyl-1-propane sodium sulfonate, 3-sulfopropyl methacrylate potassium salt, methacrylic acid, 3-[[2-(methacryloyloxy)ethyl]dimethylammonio]propionate, 2-methacryloyloxyethyl phosphorylcholine, [2-(methacryloyloxy)ethyl]dimethyl-(3-sulfopropyl) ammonium hydroxide, 3-[[2-(acryloyloxy)ethyl]dimethyl-ammonio]propionate, 2-acryloyloxyethyl phosphorylcholine, [2-(acryloyloxy)ethyl]dimethyl-(3-sulfopropyl) ammonium hydroxide, N-(2-propynyl)-acrylamide, N-(3-azidopropyl)-acrylamide, N-(3-azidopropyl)-methacrylamide, and vinyl acetate, and
a third co-monomer selected from an acryloyl or methacryloyl monomer comprising a functional group capable of reacting with an agent-containing molecule, and an acryloyl or methacryloyl monomer comprising an agent conjugated thereto.

A skilled person would appreciate that N-(2-hydroxypropyl)methacrylamide forms water-soluble, biocompatible, non-immunogenic and non-toxic polymers that are suitable as carriers for agents for biomedical applications.

In one set of embodiments, when the first co-monomer is N-(2-hydroxypropyl)methacrylamide, the second co-monomer is a monomer belonging to a class selected from acrylic acid, methacrylic acid, acrylate, methacrylate, acrylamide and vinyl ester.

In one form, when the first co-monomer is N-(2-hydroxypropyl)methacrylamide, then the second co-monomer is selected from 2-hydroxyethyl acrylate, 2-methoxyethyl acrylate, 2-(diethylene glycol) ethyl acrylate, poly(ethylene glycol) acrylate, poly(ethylene glycol) methacrylate, poly(ethylene glycol) methyl ether acrylate, poly(ethylene glycol) methyl ether methacrylate, N-acryloylamido-ethoxyethanol, N,N-dimethylacrylamide, N,N-diethylacrylamide, N-(2-hydroxyethyl) acrylamide, acrylamide, N-acryloylmorpholine, N-propyl acrylamide, N-isopropyl acrylamide, di(ethylene glycol) methyl ether methacrylate, 2-hydroxyethyl methacrylate, 2-(dimethylamino) ethyl acrylate, 2-(diethylamino) ethyl acrylate, 3-(dimethylamino) propyl acrylate, (3-acrylamidopropyl) trimethylammonium chloride, 2-aminoethyl methacrylate hydrochloride, 2-carboxyethyl acrylate, acrylic acid, N-carboxyethyl acrylamide, 2-acrylamido-2-methyl-1-propane sodium sulfonate, 3-sulfopropyl methacrylate potassium salt, methacrylic acid, N-acryloxysuccinide, 3-[[2-(methacryloyloxy)ethyl]dimethylammonio]propionate, 2-methacryloyloxyethyl phosphorylcholine, [2-(methacryloyloxy)ethyl]dimethyl-(3-sulfopropyl) ammonium hydroxide, 3-[[2-(acryloyloxy)ethyl]dimethyl-ammonio]propionate, 2-acryloyloxyethyl phosphorylcholine, and [2-(acryloyloxy)ethyl]dimethyl-(3-sulfopropyl) ammonium hydroxide.

In one set of embodiments, the copolymer backbone comprises polymerised residues of N-(2-hydroxypropyl)methacrylamide and a second co-monomer selected from the group consisting of N-acryloylmorpholine, N-isopropylacrylamide, poly(ethylene glycol) methyl ether acrylate and poly(ethylene glycol) methyl ether methacrylate, preferably N-acryloylmorpholine, and N-isopropylacrylamide.

In a particular set of embodiments, the linear copolymer backbone of the polymer conjugate comprises polymerised residues derived from:
a first co-monomer which is N-(2-hydroxypropyl)methacrylamide;
a second co-monomer selected from N-acryloylmorpholine, and N-isopropyl acrylamide; and
a third co-monomer selected from N-acryloxysuccinimide and an acrylate monomer comprising an agent conjugated thereto.

An example of an acrylate monomer-agent conjugate is shown in Formula (III) described herein, wherein R^{c} is H and X is O in these formula. The monomer-agent conjugate has an agent conjugated to the acryloyl moiety of the monomer. The conjugated agent will form a pendant group of the linear copolymer backbone following polymerisation of the monomer and its incorporation into the copolymer.

In a specific embodiment, the linear copolymer backbone of the polymer conjugate is a terpolymer. An exemplary terpolymer consists of polymerised residues derived from:
a first co-monomer which is N-(2-hydroxypropyl)methacrylamide;
a second co-monomer selected from N-acryloylmorpholine, and N-isopropyl acrylamide; and
a third co-monomer selected from N-acryloxysuccinimide and an acrylate monomer comprising an agent conjugated thereto.

In one set of embodiments, the copolymer backbone comprises polymerised residues of N-(2-hydroxypropyl)methacrylamide and N-isopropylacrylamide as co-monomers. Advantageously, it has been found that a polymer conjugate having a linear statistical copolymer backbone comprising residues derived from these monomers as part of the copolymer exhibit a higher than expected plasma concentration following administration of the polymer conjugate in vivo.

An advantage of a polymer conjugate comprising a linear, aliphatic, statistical copolymer backbone derived from at least three different ethylenically unsaturated monomers is that the composition of the copolymer can be adjusted to tailor the properties of the polymer conjugate. For instance, the type of ethylenically unsaturated groups in the co-monomers, the type of substituent groups present on the co-monomers, and the relative quantity of each co-monomer, can each influence properties of the polymer conjugate, such as hydrophilicity, hydrodynamic volume and pharmacokinetic properties. Thus adjustments can be made to the composition of the copolymer by adjusting the types of monomer from which the copolymer is derived. In turn, this can provide an avenue for adjusting the properties of the polymer conjugate and thus tailoring the polymer conjugate for specific applications (e.g. the delivery of specific agents)

For example, it has been observed that the composition of the linear copolymer can influence the hydrodynamic volume of the copolymer and this in turn can affect the pharmacokinetics of a polymer conjugate comprising the copolymer. Linear copolymer backbones exhibiting larger hydrodynamic volumes may be cleared at slower rates and thus have a longer retention in vivo than those exhibiting smaller hydrodynamic volumes. Polymer conjugates comprising a linear copolymer backbone derived from at least three different ethylenically unsaturated monomers as described herein can advantageously be tailored to exhibit different hydrodynamic volumes through the selection of different co-monomers used in formation of the copolymer backbone.

As an example, it has been observed that a copolymer comprising polymerised residues derived from N-(2-hydroxypropyl)methacrylamide (HPMA) and N-isopropyl acrylamide (NIPAM) as predominant components of the copolymer can exhibit a hydrodynamic volume that is larger than expected for the copolymer's size and composition at physiological temperature (approximately 37° C.). Without wishing to be limited by theory, it is believed this unexpected hydrodynamic volume may be related to the presence of a combination of HPMA and NIPAM in the copolymer, where HPMA may be influencing the lower critical solubility temperature (LCST) of NIPAM. NIPAM is used in the preparation of temperature sensitive, water-swellable polymers, and can be combined with other water-soluble monomers to modify the lower critical solubility temperature (LCST) of the polymer. However, p(NIPAM) polymers generally shrink at about 37° C., and thus copolymers comprising NIPAM may expected to undergo shrinkage as temperature is increased from room temperature (approximately 20° C.), thereby forming polymers of reduced hydrodynamic volume in vivo. However, the finding that a copolymer comprising polymerised residues derived from HPMA and NIPAM exhibits an increase in hydrodynamic volume at 37° C. is unexpected. The change in hydrodynamic volume can influence the pharmacokinetics of the polymer conjugate and thus provide for a longer or shorter circulation half-life for the conjugate in vivo.

A further benefit that may associated with a copolymer derived from at least three different co-monomers is the greater flexibility in modifying the composition of the copolymer due to the larger number of potential monomer combinations that are possible when at least three different monomers are employed. This compares to copolymers formed with less than three co-monomers, where fewer monomer combinations would potentially be available and thus there could be less flexibility in making compositional changes in the copolymer.

Additionally, when the linear copolymer backbone comprises polymerised residues that are derived from three different co-monomers, residues derived from two of the three co-monomers may be present in comparatively larger amounts compared to those derived from the third co-monomer. Thus the properties of the polymer conjugate may be largely influenced by the two co-monomers, which are predominant components of the copolymer backbone. Accordingly, the two co-monomers may be selected to impart desired physical properties to the polymer conjugate. Residues in the copolymer derived from the third co-monomer can provide a site for conjugation of an agent and thus, depending on the desired loading of agent, a relatively small amount of polymerised resides derived from the third co-monomer may be present. The ethylenically unsaturated group of the third co-monomer may be selected to have a reactivity that promotes a random distribution of the third co-monomer in the copolymer backbone. In this manner, a random distribution of conjugated agent may be afforded along the length of the copolymer chain.

Polymer conjugates of the invention, which comprise a linear, aliphatic copolymer backbone composed of carbon atoms, also advantageously exhibit stability in vivo. That is, the aliphatic copolymer backbone is not degraded or broken down in the physiological environment but is instead cleared as a whole polymer. In limiting the breakdown of the copolymer backbone, issues associated with potential accumulation or toxicity, which might be associated with smaller polymer fragments, can be at least be reduced or avoided. Furthermore, from an ADMET (absorption, distribution, metabolism, excretion, toxicity) perspective, whole structure clearance of an intact polymeric molecule is more predictable than that of polymer fragments. These benefits can therefore be of assistance for obtaining regulatory approval from relevant regulatory authorities.

The copolymer backbone may be of any suitable size or molecular weight. Preferably, the copolymer backbone is about 1 kDa or larger. In one preference, the copolymer backbone has a molecular weight of no more than about 40 kDa, preferably a molecular weight in a range of from about 15 to 35 kDa. Suitably, the copolymer backbone is of a size that aids in increasing the retention of the conjugated agent and the binding moiety in vivo.

In some embodiments, the copolymer backbone is of a size that is large enough to promote acceptable circulating half-life for the polymer conjugate to allow for accumulation, yet is small enough to be capable of renal clearance after delivery.

Linear, aliphatic, copolymer backbones described herein may be prepared in any suitable manner. A suitable synthetic method used to produce the copolymer backbones provided herein is free radical polymerisation.

A skilled person would understand that free radical polymerisation of monomers involves the propagation of a free radical species though an ethylenically unsaturated moiety of different co-monomers. This results in the formation of a carbon-carbon bond that covalently links the different co-monomers together.

In one set of embodiments, the copolymer backbone that is derived from at least three different ethylenically unsaturated monomers is formed using a living radical polymerisation process. In certain embodiments, Reversible Addition-Fragmentation chain Transfer (RAFT) is used to synthesise the copolymer backbone of the polymer conjugates of the invention. One advantage associated with copolymer backbones prepared using living radical polymerisation processes such as RAFT is that the resultant polymer has a narrow polydispersity index (PDI). In some particular embodiments, the copolymer backbone of the polymer conjugate described herein has a polydispersity index of no more than about 1.5, preferably no more than about 1.3.

Additionally, a copolymer backbone formed using RAFT polymerisation will comprise end groups derived from the RAFT agent used to form the polymer. The RAFT end groups may be removed or modified to generate a terminal functional group at one or both ends of the linear polymer, which may be used to tether a binding moiety to an end of the linear copolymer chain. For example, removal of a RAFT end group may provide a terminal thiol functional group at an end of the copolymer backbone, which can be utilised for conjugation of a binding moiety or an agent. Some examples of RAFT agents that may be employed for formation of the linear copolymer backbone are described in Macromolecules, 2012, 45, 5321-5342.

The polymer conjugate of the invention also comprises a binding moiety conjugated to an end of the linear, aliphatic, statistical copolymer backbone. The binding moiety is conjugated to one selected from the alpha (a) end and the omega (w) end of the copolymer.

An agent (such as a therapeutic or diagnostic agent) is also conjugated to the copolymer backbone. The agent may be conjugated to an end of the copolymer backbone, opposing the binding moiety, and/or to a pendant group of one or more monomeric units of the copolymer backbone.

In certain embodiments, the polymer conjugate described herein comprises a binding moiety coupled to the alpha end (α-end) of the copolymer backbone. In such embodiments, the polymer conjugate further comprises an agent, which may be coupled to the omega end (ω-end) of the copolymer backbone and/or to a pendant group of a monomeric unit of the copolymer backbone

A “binding moiety” is a group with a specific affinity for a target compound, such as a cell surface epitope associated with a specific disease state. In some embodiments, binding moieties recognise a cell surface antigen or bind to a receptor on the surface of the target cell.

The binding moiety can enhance the bio-distribution properties of the polymer conjugate to which it is attached, to improve cellular distribution and cellular uptake of the conjugate, by enhancing the association of the conjugate with a target cell or tissue.

It is believed that by attaching the binding moiety to an end of the linear copolymer backbone, the binding moiety is less hindered by polymer steric bulk and thus is more readily accessible for binding to a target site, such as a target antigen or receptor.

Furthermore, by attaching the binding moiety to an end of the copolymer backbone, efficient conjugation of the binding moiety to the backbone can be achieved. This is because attachment of the binding moiety can be facilitated when a terminal functional group at an end of the linear copolymer is reacted with a suitable binding moiety containing compound. In comparison, chemical reactions that attach a binding moiety at a position in the middle of the linear copolymer backbone can be less efficient due to steric factors influencing the effectiveness of the reaction.

The binding moiety of the polymer conjugate may be selected from a range of suitable groups useful for targeting cellular or tissue sites. A skilled person would be able to select a particular binding moiety that is capable of targeting a particular cellular or tissue site of interest.

In some embodiments, the binding moiety is a protein. An exemplary protein is an antibody.

In some particular embodiments, the binding moiety is selected from the group consisting of an antibody, an antibody fragment and an antigen binding fragment. In a specific embodiment, the binding moiety is a Fab′ fragment.

Full length intact antibodies and antibody fragments may be used as a binding moiety in the polymer conjugate of the invention.

A skilled person would understand that antibody fragments may be produced by digestion of an antibody with various peptidases or chemicals. Thus, for example, pepsin digests an antibody below the disulfide linkages in the hinge region to produce F(ab′)₂, a dimer of Fab which itself is a light chain joined to VH-CH1 by a disulfide bond. The F(ab′)₂ may be reduced under mild conditions to break the disulfide linkage in the hinge region thereby converting the F(ab′)₂ dimer into an Fab′ fragment. The Fab′ fragment is essentially a Fab fragment with part of the hinge region that contains reduced cysteine-residue thiols. The antibody fragment can also be engineered and expressed directly, as a Fab, scFv or any other well understood antibody fragment.

Attachment of the binding moiety to an end of the copolymer backbone is achieved in any suitable manner, e.g., by any one of a number of bioconjugation chemistry approaches.

In one embodiment, when the binding moiety is an antibody fragment such as a Fab′ fragment, the binding moiety is conjugated to the copolymer backbone via a thiol residue on the antibody fragment.

In one set of embodiments the binding moiety is conjugated to the copolymer backbone via a linker. Preferably, the linker conjugating the binding moiety to the copolymer is a biologically stable linker. It can be important for the copolymer backbone and the binding moiety to remain conjugated to each other in a biological environment as insufficient stability can lead to premature or unwanted loss or release of the binding moiety and hence loss of the conjugate's targeting ability. The biostability of the copolymer-binding moiety conjugate can be dependent on the chemistry of the linker that bridges the copolymer backbone and the binding moiety.

As used herein, a “linking moiety” or a “linker” is a chemical bond or a multifunctional (e.g., bifunctional) residue which is used to link a molecule, such as a binding moiety or an agent (e.g. a therapeutic or diagnostic agent) to the copolymer backbone of the conjugate.

The linker may be biodegradable (i.e. cleavable) or non-biodegradable (i.e. biostable or non-cleavable). Cleavable linkers can be hydrolysable, enzymatically cleavable, pH sensitive, photolabile, or disulphide linkers, among others.

Linkers useful for the present invention may be derived from a variety of compounds. Linkers used in click chemistry, maleimide chemistry and NHS-esters can be used. The linkers can be derived from compounds which can provide an amide, ester, ether, thio-ether, carbamate, urea, amine, triazole, disulphide, hydrazone, or other suitable linkage for conjugating a molecule (such as a binding moiety or agent) to the copolymer backbone.

In some embodiments, linker compounds for conjugating a binding moiety to the copolymer backbone may provide biodegradable or non-biodegradable (i.e. biostable) linkage. Biodegradable linkages may include amide, ester, carbamate, urea or amine moieties. Generally acceptable biostable linkages may include triazole, ether and thio-ether moieties.

In some embodiments, the binding moiety is conjugated to the copolymer backbone via a linker comprising a thio-ether moiety.

In some embodiments, the binding moiety is conjugated to the copolymer backbone via a linker comprising a moiety of formula (I):

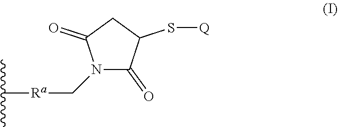

where:
Q represents the binding moiety;
R^{a} represents the remainder of the linker; and
[symbol] represents a site of attachment to an end of the copolymer backbone.

In particular embodiments, the binding moiety is conjugated to the copolymer backbone via a linker comprising a moiety of formula (II):

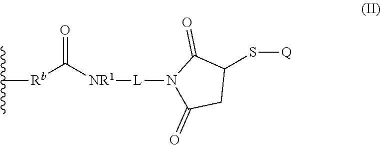

where:
Q represents the binding moiety;
R¹ is H or C1-C4 alkyl;
R^{b} is a bond or C2 alkyl;
L is a linking moiety; and
[symbol] represents a site of attachment to an end of the copolymer backbone.

In some specific embodiments of formula (II), the linking moiety (L) comprises a C2-C3 polyether. In one preference, L comprises poly(ethylene glycol). In one example, L comprises a poly(ethylene glycol) moiety of the following structure:

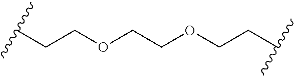 [SMILES: *CCOCCOCC*]

A linker of formula (I) or (II) may be formed by covalently reacting a suitably functionalised linker molecule with a terminal functional group at an end of the copolymer backbone and with a functional group present in a binding moiety. The linker then spans between and joins the copolymer backbone and the binding moiety.

In one form, a moiety of formula (I) or (II) can be formed when a thiol functional group (e.g. thio alkyl) reacts with a maleimido moiety to generate an S-maleimido group of the following structure:

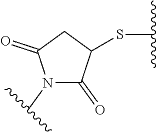 [SMILES: *SC1CC(=O)N(*)C1=O]

In some embodiments, a linker of formula (I) or (II) may be derived from a suitably difunctionalised linker molecule.

In one embodiment, the copolymer backbone comprises a terminal thiol functional group and the linker molecule is a difunctional compound having a functional group adapted to covalently react with the terminal thiol functional group on the copolymer. The other functional group of the difunctional compound may be adapted to covalently react with a functional group present in a binding moiety.

In one set of embodiments, the linker molecule is a difunctional compound comprising two unsaturated functional groups. Such a difunctional molecule may be a bismaleimide as shown below:

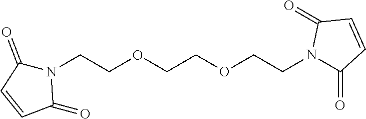 [SMILES: O=C1C=CC(=O)N1CCOCCOCCN1C(=O)C=CC1=O]

When reacting a difunctional compound to form a linker, an unsaturated functional group (i.e. maleimido moiety) can participate in a Michael addition with a terminal thiol functionality at an end of the copolymer backbone to attach the linker to the backbone. Once the linker is attached, the remaining unsaturated functional group (i.e. a maleimido moiety) may then covalently react with a binding moiety comprising a thiol residue to thereby conjugate the binding moiety to the copolymer backbone via the thiol. The reaction between the binding moiety and the linker forms a thio-ether moiety. In one preference, a linker comprising a thio-ether moiety may be of formula (I) or (II).

In another set of embodiments, a linker may be introduced by covalently reacting a terminal functional group on the copolymer backbone with an intermediate compound to form an intermediate species, which can then be chain-extended to install a functional group suitable for reacting with a binding moiety at the end of the copolymer chain. An example is shown below, where the copolymer backbone is reacted with a diamine compound to form an intermediate with an amino functionality. The amino functionality can subsequently be reacted with a maleimide-containing compound to introduce a maleimide functionality for reaction with a thiol residue on a binding moiety:

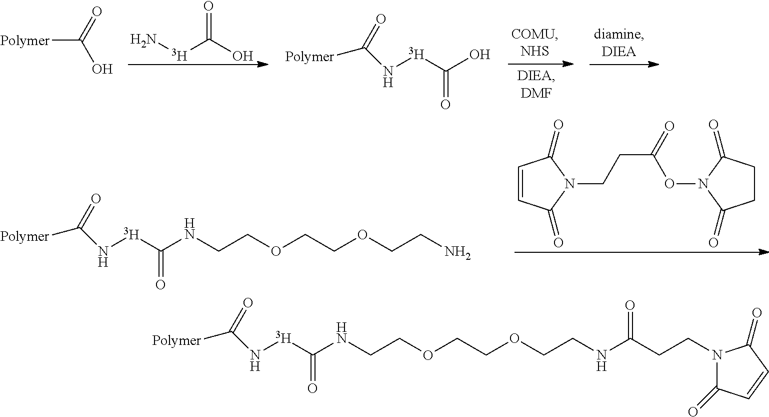

Other intermediate compounds and maleimide-containing compounds suitable for introducing linkers for conjugation of a binding moiety to the copolymer backbone would be known to a skilled person.

Some examples of moieties that provide a maleimide functional group at an end of the linear copolymer backbone for conjugation with a binding moiety are shown below:

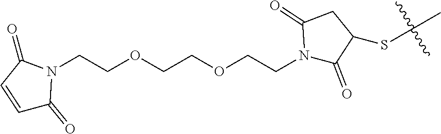 [SMILES: *SC1CC(=O)N(CCOCCOCCN2C(=O)C=CC2=O)C1=O]

(A) bis-maleimide installed onto a terminal thiol of a linear copolymer backbone

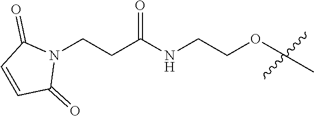 [SMILES: *OCCNC(=O)CCN1C(=O)C=CC1=O]

(B) maleimide-PEG installed on a terminal carboxylic acid of linear copolymer backbone

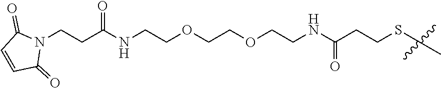 [SMILES: *SCCC(=O)NCCOCCOCCNC(=O)CCN1C(=O)C=CC1=O]

(C) maleimide installed onto a terminal thiol of a linear copolymer backbone via a PEG-amide linker

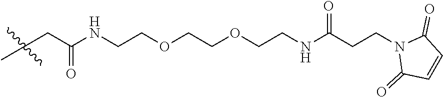 [SMILES: *CC(=O)NCCOCCOCCNC(=O)CCN1C(=O)C=CC1=O]

(D) maleimide installed onto a terminal carboxylic acid of linear copolymer backbone via a PEG amide linker

A skilled person would appreciate that there are many other functional groups that will selectively react with thiols that have been shown to work well in the presence of proteins (e.g. vinyl sulfone, pyridyl disulphide, haloacetyl (e.g. bromoacetyl or iodoacetyl)). Any one of these chemistries is robust and fast, unambiguous, produces a stable product and is well understood and acceptable for biological applications.

As described herein, the polymer conjugate also comprises an agent conjugated to the linear, aliphatic, statistical copolymer backbone. The agent may be conjugated to an end of the copolymer backbone and/or to a pendant group of a monomeric unit of the copolymer backbone.

In one embodiment of polymer conjugates described herein, the agent is conjugated to an end of the linear copolymer backbone. In such embodiments it is a proviso that the agent and binding moiety are conjugated to different ends of the copolymer. That is, if the conjugate comprises a binding moiety conjugated to the ca-end of the backbone, then the agent is coupled to the co-end of the copolymer backbone, and vice versa.

In another embodiment, the agent is conjugated to and pendant from the copolymer backbone. The agent is therefore attached to and pendant from a polymerised monomeric unit of the copolymer backbone. In such embodiments, the agent can be covalently conjugated via a functional group that is pendant from the copolymer backbone.

Polymer conjugates of the invention comprise at least one agent and may comprise a plurality of agents. When a plurality of agents is present, they may each be of the same type or of different types of agent.

When the polymer conjugate comprises a plurality of agents, each of the agents may be pendant from the copolymer backbone. Alternatively, one of the plurality of agents may be conjugated to an end of the copolymer backbone, while the remainder of the plurality of agents are pendant from the copolymer backbone.

The agent or agents conjugated to the linear copolymer backbone may be selected from therapeutic agents and diagnostic agents. However, the present invention is not limited for use with any particular agent and a wide variety of different agents may be conjugated to the linear copolymer backbone.

Polymer conjugates of the invention may comprise a combination of different agents, such as a combination of two or more different therapeutic agents or diagnostic agents, or combinations of therapeutic and diagnostic agents.

In one set of embodiments, polymer conjugates described herein comprise a diagnostic agent conjugated to the copolymer backbone. Diagnostic agents are compounds or molecules that assist in the diagnosis of a disease or disorder. In one form, the polymer conjugate comprises a diagnostic agent, which may be a protein or peptide.

As used herein with reference to a diagnostic agent, the terms “peptide” and “protein” are used to refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types.

In one set of embodiments, diagnostic agents may be selected from the group consisting of a receptor, a ligand and an enzyme.

Diagnostic agents may be imaging agents. Imaging agents can provide for contrast in one or more imaging techniques, including but not limited to: photoacoustic imaging, fluorescence imaging, ultrasound, PET, CAT, SPECT and MRI.

Diagnostic agents may be fluorophores or dyes.

In one set of embodiments, polymer conjugates described herein comprise a therapeutic agent conjugated to the copolymer backbone. Therapeutic agents include drugs and other molecules with pharmaceutical activity designed for therapeutic purposes. Therapeutic agents may also include prodrugs. A prodrug is an inactive form of a drug that is convertible into therapeutically active form in vivo.

Therapeutic agents may be selected from a wide range of agents. Examples of therapeutic agents may include hydrophilic or hydrophobic drugs.

In one preference, the therapeutic agent is a small molecule (i.e. a molecule having a molecular weight of no more than about 1000 Da).

An exemplary small molecule may be an anti-neoplastic (i.e. anti-cancer) agent. Examples of anti-cancer agents include, without limitation, monomethyl auristatin E (MME), methotrexate, trimetrexate, adriamycin, taxotere, doxorubicin, 5-flurouracil, vincristine, vinblastine, pamidronate disodium, cyclophosphamide, epirubicin, megestrol, tamoxifen, paclitaxel, docetaxel, capecitabine, and goserelin acetate.

The polymer conjugate of the invention is capable of solubilising small molecule cytotoxic drugs that may be insoluble or poorly soluble in water due their highly aromatic chemical structure and/or lipophilic properties.

Furthermore, many low molecular weight small molecule drugs are quickly cleared from the body, leaving the circulation in minutes. The polymer conjugates of embodiments of the invention are capable of remaining in the circulation for a longer period of time, leading to a potential increase in drug uptake at a targeted site. The inclusion of a binding moiety in the polymer conjugate can give rise to an increase in targeting for a desired tissue site by receptor-mediated delivery.

The agent or agents forming part of the polymer conjugates of the invention may be conjugated to the linear, aliphatic copolymer backbone via a covalent bond or via a linker.

Linkers used for conjugation of one or more agents may be biodegradable or non-biodegradable (i.e. biostable) linkers. Examples of biodegradable and non-biodegradable linkers are described herein.

Linkers described herein above for conjugating a binding moiety to the linear copolymer backbone can also be used to conjugate an agent to the copolymer backbone.

In one set of embodiments, when the polymer conjugate comprises a diagnostic agent, the diagnostic agent may be conjugated to the linear, aliphatic copolymer backbone via a non-biodegradable linker. A non-biodegradable linker is considered to be non-cleavable or generally biostable in a biological environment. The use of a non-biodegradable linker may be preferred to limit loss of the diagnostic agent from the conjugate in the vicinity of the targeted cell or tissue.

In one embodiment, a non-biodegradable linker may comprise a triazole moiety, which is not susceptible to biodegradation or cleavage in vivo. A skilled person would appreciate that a triazole moiety is formed when alkynyl and azido functional groups covalently react under click chemistry conditions. Thus a diagnostic agent may comprise an alkynyl or azido functional group, which is capable of reacting with a complementary alkynyl or azido functional group that is pendant from the linear copolymer backbone under click chemistry conditions, to thereby form a triazole moiety that links the diagnostic agent to the copolymer backbone.

In one set of embodiments, when the polymer conjugate comprises a therapeutic agent, the therapeutic agent may be conjugated to the linear, aliphatic copolymer backbone via a biodegradable linker. A biodegradable linker can be advantageous as it can be susceptible to breakdown or cleavage under certain conditions and thereby facilitate release of the therapeutic agent in response to an appropriate stimulus once the polymer conjugate reaches a desired site in vivo.

In one preference, the therapeutic agent is conjugated to the copolymer backbone via a biodegradable linker that is enzymatically cleavable. An “enzymatically cleavable linker” refers to a linkage that is subject to degradation by one or more enzymes. A number of enzymatically cleavable linkers may be used, and such linkers would be known to a skilled person. In one embodiment, the biodegradable linker is an enzymatically cleavable linker comprising a moiety selected from the group consisting of valine-citrulline-para-aminobenzoic acid (Val-Cit-PABA), valine-alanine (Val-Ala), and phenylalanine-lysine (Phe-Lys). Enzymatically cleavable linkers have been found to facilitate the desired release of a therapeutic agent in a potent, pharmaceutically active form.

In one particular embodiment, a polymer conjugate according to the present invention comprises:
a linear, aliphatic, statistical terpolymer backbone having two ends;
a Fab′ fragment conjugated to an end of the copolymer backbone; and
at least one agent conjugated to the copolymer backbone,
wherein the agent is conjugated to an end of the terpolymer backbone or is conjugated to and pendant from the terpolymer backbone, with the proviso that when conjugated to an end of the backbone then the agent and the Fab′ fragment are conjugated to different ends of the terpolymer backbone.

As used herein a “terpolymer” is a copolymer derived from three different ethylenically unsaturated monomers. Thus the terpolymer has polymerised residues derived from the three co-monomers.

In one preference, the three different ethylenically unsaturated monomers from which the terpolymer backbone is derived are each hydrophilic monomers.

In some embodiments, the terpolymer is suitably derived from three different ethylenically unsaturated monomers, wherein each monomer has a different ethylenically unsaturated group.

In one preference, the three monomers having different ethylenically unsaturated groups belong to different classes of monomer selected from acrylic acid, methacrylic acid, acrylate, methacrylate, acrylamido, methacrylamido and vinyl ester. Some specific examples of monomers belonging to these classes of monomers are described herein above.

In one preference, the agent is a therapeutic agent. In such embodiments, the therapeutic agent may be conjugated to the terpolymer backbone via a biodegradable linker, such as an enzymatically cleavable linker as described herein.

Polymer conjugates of the present invention may be prepared using a variety of different synthetic approaches.

In one embodiment, the polymer conjugate may be prepared by first synthesising a linear, aliphatic, statistical copolymer, then conjugating a binding moiety and an agent to the pre-formed copolymer. The binding moiety may be conjugated to the copolymer first, followed by the agent, or vice versa. The binding moiety and agent may be conjugated to the copolymer via appropriate functional groups on the copolymer.

Thus in a second aspect the present invention provides a process for preparing a biocompatible, hydrophilic polymer conjugate, the process comprising the steps of:
(a) polymerising a monomer composition comprising at least three different ethylenically unsaturated monomers under conditions of free radical polymerisation to form a linear, aliphatic, statistical copolymer backbone having two ends and comprising a first functional group for conjugating a binding moiety at a first end of the copolymer backbone, and a second functional group for conjugating an agent at a position selected from the second end of the copolymer backbone and pendant from the copolymer backbone;
(b) covalently reacting the first functional group with a binding moiety-containing molecule to conjugate the binding moiety to the first end of the copolymer backbone; and
(c) covalently reacting the second functional group with an agent-containing molecule to conjugate the agent to the copolymer backbone at a position selected from the second end of the copolymer backbone and pendant from the copolymer backbone.

A skilled person would appreciate that the order of steps (b) and (c) in the above process may be reversed, such that the agent may be conjugated to the copolymer backbone prior to conjugation of the binding agent.

It is a requirement that copolymer backbones formed in accordance with processes described herein do not comprise a block copolymer. Thus the copolymer backbone of the polymer conjugate of the invention is not a block copolymer. Suitably, the copolymer backbone comprises a statistical copolymer.

When a functional group is at an end of the copolymer backbone, the functional group is considered to be a terminal functional group.

In one preference, the different ethylenically unsaturated co-monomers in the monomer composition of the second aspect have different ethylenically unsaturated groups. The co-monomers may belong to different classes of monomer as described herein.

Polymerisation of the monomer composition suitably takes place under conditions of free radical polymerisation. In one embodiment, the monomer composition is polymerised by a process of living free radical polymerisation, preferably reversible-addition-fragmentation-chain transfer (RAFT) polymerisation.

Using a free radical polymerisation process, suitable co-monomers and optionally, an initiator as a source of free radicals are combined and triggered to react under conditions of free radical polymerisation. In certain instances, the process for forming the copolymer backbone involves forming a monomer composition comprising at least three different ethylenically unsaturated monomers and subjecting the monomer composition to free radical polymerisation conditions. The free radical polymerisation may be carried out in any suitable manner, including, e.g., in solution, dispersion, suspension, emulsion or bulk.

The monomer composition may comprise one or more additional components that facilitate the free radical polymerisation reaction. For example, the monomer composition can comprise a suitable solvent for solubilising the monomers contained therein. The solvent may be an organic solvent or an aqueous solvent. Mixtures of solvents may be used. The choice of solvent may depend on the type of co-monomers used to form the copolymer and the polymerisation conditions (including RAFT agent) employed.

When RAFT polymerisation is employed to prepare the linear, aliphatic copolymer backbone, a RAFT agent is selected to facilitate the polymerisation. A range of RAFT agents may be employed and the selection of an appropriate RAFT agent might depend on the monomers being polymerised and the type of RAFT end groups that could be carried on the resulting polymer. One example of a RAFT agent that is suitable for the preparation of the copolymer backbone of polymer conjugates of the invention is 4-cyano-4-(phenylcarbonothioylthio)pentanoic acid. A skilled person would be able to select a suitable RAFT agent for formation of a copolymer of desired composition and functionality.

The pre-formed copolymer backbone prepared in accordance with the above process comprises at least two functional groups and these may be referred to herein as a first functional group and a second functional group.

The first functional group is for conjugating a binding moiety and is a terminal functional group and is situated at a first end of the copolymer backbone. The second functional group is for conjugating an agent and may either be a terminal functional group situated at a second end of the copolymer backbone or be a pendant functional group. By being “pendant”, the functional group does not directly form part of the chain of carbon atoms of the copolymer backbone.

In some embodiments, the linear copolymer backbone will comprise a terminal functional group at one end or both of ends of the copolymer chain. The terminal functional group or groups are capable of participating in covalent reactions to conjugate a binding moiety to a first end of the copolymer, and optionally, to also conjugate an agent to a second end of the copolymer.

In some other embodiments, the linear copolymer backbone will comprise a terminal functional group at one end of the polymer chain for conjugating a binding moiety, and also comprise one or more pendant functional groups.

In some other embodiments, the linear copolymer backbone will comprise two terminal functional groups, one at each end of the copolymer chain, and will also comprise one or more functional groups pendant from the copolymer chain. One of the terminal functional groups is for conjugating a binding moiety. The other terminal functional group and/or the pendant functional group or groups are for conjugating with an agent, such as therapeutic or diagnostic agent.

Pendant functional groups may be capable of participating in hydrogen bonding interactions with water and in this way, help to promote the hydrophilicity of the copolymer backbone and hence the polymer conjugate. Pendant functional groups may also be capable of participating in covalent reactions that facilitate conjugation and attachment of an agent to the copolymer backbone to form the polymer conjugate.

The first functional group and the second functional group of the linear copolymer backbone may be of the same type or of different types.

The first functional group may be derived from a RAFT end group, which is introduced when a RAFT polymerisation process is used to form the linear copolymer. Alternatively, the functional group may be formed upon removal or conversion of a RAFT end group.

For example, a thiocarbonylthio RAFT end group can be converted into a thiol functionality. A skilled person would understand that a linear polymer prepared using a RAFT polymerisation process can contain two RAFT end groups and either one of the RAFT end groups may form, or be converted into, a functional group that is suitable for conjugation with a binding moiety.

In one set of embodiments, the linear, aliphatic, statistical copolymer backbone comprises a thiol terminal functional group and a carboxylic acid terminal functional group, wherein the thiol and carboxylic acid functional groups are at different ends of the linear copolymer chain.

Some other types of terminal functional groups may be generated from RAFT end groups. Examples of other types of terminal functional groups include but are not limited to dithiocarbamate, succinimidyl, azido, alkynyl, maleimido, and cyclic acetal functional groups. A terminal functional group selected from the above may be present in the linear copolymer backbone in addition to a terminal thiol functional group. Such terminal functional groups will be at a different end of the copolymer chain to the terminal thiol.

A skilled person would appreciate that different RAFT agents may generate different types of functional groups at the end or ends of the linear copolymer chain. Synthetic methodologies for coupling binding moieties and agents (if desired) to one or more ends of the linear copolymer backbone may be selected to suit the type of functional group present at a terminus of the copolymer and/or to suit functional groups present in a particular binding moiety or agent.

Conjugation of the binding moiety to the linear copolymer can proceed by covalently reacting the first functional group at an end of the copolymer chain with a binding-moiety containing molecule. This results in direct coupling of the binding moiety to the end of the copolymer.

Alternatively, the first functional group may be reacted with a linker molecule to couple a linker to the linear copolymer via the functional group. The linker molecule can in turn, have a terminal functionality that is available to covalently react with a binding moiety-containing molecule to conjugate a binding moiety to the linear copolymer via the intermediate linker. In one preference, the linker molecule provides a non-biodegradable linker that couples the binding moiety to the linear copolymer. Examples of non-biodegradable linkers are described herein.

Particular linker molecules for conjugating a binding moiety to the copolymer backbone are maleimide-containing linker molecules, which can react with the first functional group at the first end of the copolymer backbone to install a maleimide functional group at the first end of the copolymer. Some examples of maleimide-containing linkers that can be generated following reaction of the first functional group with a linker molecule are shown below:

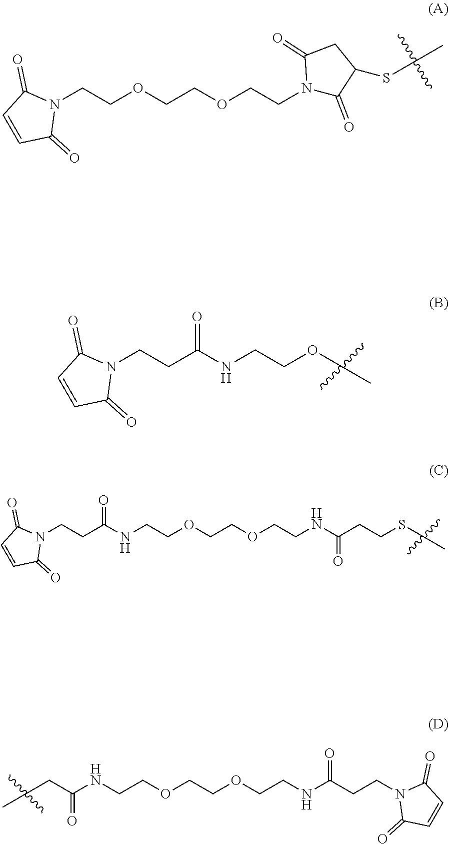 [SMILES: *SC1CC(=O)N(CCOCCOCCN2C(=O)C=CC2=O)C1=O ; *OCCNC(=O)CCN1C(=O)C=CC1=O ; *SCCC(=O)NCCOCCOCCNC(=O)CCN1C(=O)C=CC1=O ; *CC(=O)NCCOCCOCCNC(=O)CCN1C(=O)C=CC1=O]

A range of binding moiety-containing molecules may be used. In some embodiments, the binding moiety-containing molecule comprises a protein, preferably an antibody, an antibody fragment or an antigen binding fragment. In one embodiment, the binding moiety-containing molecule is Fab′-SH.

When situated at an end of the linear copolymer, the second functional group may also be derived from a RAFT end group.

Alternatively, when the second functional group is a pendant functional group, the second functional group may be introduced by adding and polymerising an appropriately functionalised co-monomer in the monomer composition in order to form a functionalised linear copolymer. Exemplary pendant functional groups may be hydroxyl, amino, carboxyl, alkynyl, azido and succinimido, preferably succinimido.

Conjugation of the agent to the linear copolymer can proceed by covalently reacting the second functional group (situated at an end of the copolymer and/or pendant from the copolymer) directly with an agent-containing molecule. In such embodiments, the agent-containing molecule can comprise a functional group that is complementary to the second functional group of the linear copolymer, such that reaction between the functional groups forms a covalent bond that results in coupling of the agent to the copolymer backbone.

In one embodiment, the agent-containing molecule comprises a diagnostic or therapeutic agent.

In some embodiments, covalent reaction of the second functional group with an agent-containing molecule may proceed via a linker. The linker may be a biodegradable (i.e. cleavable) linker or non-biodegradable (i.e. non-cleavable) linker derived from an appropriate linker molecule. Examples of biodegradable and non-biodegradable linkers are described herein.

In one embodiment, the agent-containing molecule comprises a therapeutic agent and a biodegradable linker that is coupled to the therapeutic agent. In such embodiments, the second functional group on the copolymer backbone may covalently react with a complementary functional group on the linker portion of the agent-containing molecule to covalently couple the therapeutic agent to the copolymer backbone via the biodegradable linker. A suitable biodegradable linker may be an enzymatically cleavable linker, examples of which are described herein.

Alternatively, the second functional group on the copolymer backbone may initially covalently react with a linker molecule to couple a linker to the linear copolymer via the second functional group. The coupled linker can in turn, have a terminal functionality that is available to covalently react with a complementary functional group present on an agent-containing molecule to thereby conjugate the agent to the linear copolymer via the intermediate linker. In some embodiments, the linker is a biodegradable linker, such as an enzymatically cleavable linker, examples of which are described herein.

In some embodiments, the monomer composition comprises three different ethylenically unsaturated co-monomers and polymerisation of the monomer composition produces a linear, aliphatic, statistical terpolymer comprising polymerised residues derived from the three different co-monomers.

In one embodiment of the process of the second aspect of the invention described herein, the monomer composition comprises:
(a) a first co-monomer selected from N-(2-hydroxypropyl)methacrylamide and N-(2-hydroxypropyl) acrylamide,
(b) a second co-monomer selected from 2-hydroxyethyl acrylate, 2-methoxyethyl acrylate, 2-(diethylene glycol) ethyl acrylate, poly(ethylene glycol) acrylate, poly(ethylene glycol) methacrylate, poly(ethylene glycol) methyl ether acrylate, poly(ethylene glycol) methyl ether methacrylate, N-acryloylamido-ethoxyethanol, N,N-dimethylacrylamide, N,N-diethylacrylamide, N-(2-hydroxyethyl) acrylamide, N-(2-hydroxyethyl) methacrylamide, N-[Tris(hydroxymethyl)methyl]acrylamide, acrylamide, N-acryloylmorpholine, N-propyl acrylamide, N-isopropyl acrylamide, methacrylamide, di(ethylene glycol) methyl ether methacrylate, 2-hydroxyethyl methacrylate, 2-(dimethylamino) ethyl acrylate, 2-(diethylamino) ethyl acrylate, 3-(dimethylamino) propyl acrylate, (3-acrylamidopropyl) trimethylammonium chloride, 2-aminoethyl methacrylate hydrochloride, [3-(methacryloylamino)propyl]trimethylammonium chloride, 2-carboxyethyl acrylate, acrylic acid, N-carboxyethyl acrylamide, 2-acrylamido-2-methyl-1-propane sodium sulfonate, 3-sulfopropyl methacrylate potassium salt, methacrylic acid, 3-[[2-(methacryloyloxy)ethyl]dimethylammonio]propionate, 2-methacryloyloxyethyl phosphorylcholine, [2-(methacryloyloxy)ethyl]dimethyl-(3-sulfopropyl) ammonium hydroxide, 3-[[2-(acryloyloxy)ethyl]dimethyl-ammonio]propionate, 2-acryloyloxyethyl phosphorylcholine, [2-(acryloyloxy)ethyl]dimethyl-(3-sulfopropyl) ammonium hydroxide, N-(2-propynyl)-acrylamide, N-(3-azidopropyl)-acrylamide, N-(3-azidopropyl)-methacrylamide, and vinyl acetate; and
(c) a third co-monomer which is an acryloyl or methacryloyl monomer comprising a functional group adapted to covalently react with an agent-containing molecule.

The polymerised co-monomers form polymerised residues (i.e. monomeric units) in the resultant copolymer.

The functional group of the third co-monomer may form a pendant functional group in the resultant linear, aliphatic copolymer. The pendant functional group is capable of covalently reacting with an agent-containing molecule to aid in conjugation of the agent to the copolymer backbone.

In one particular embodiment, the third co-monomer is N-acryloyloxysuccinimide (NAS).

One exemplary process for forming a linear, aliphatic, statistical copolymer backbone from at least three different co-monomers is illustrated below:

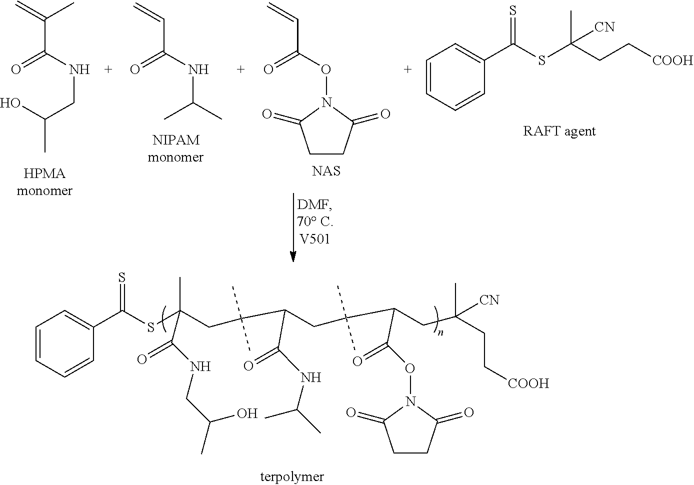

The functional group on the third co-monomer can form a pendant functional group on the resulting linear, aliphatic copolymer, which is available for conjugation of an agent, such as a diagnostic or therapeutic agent. The pendant functional group can be considered to be a second functional group of the copolymer.

The pendant functional group that is provided on the copolymer backbone after incorporation of the third co-monomer is thus capable of reacting with an agent-containing molecule for loading of the agent onto the backbone.

Following conjugation of the agent to one or more copolymer pendant functional groups, any residual pendant functional groups (i.e. not conjugated with an agent) may be reacted to convert the pendant functionality into a non-reactive moiety, which may be more compatible with a biological environment. For example, residual succinimido functionalities that are pendant from the linear copolymer chain may be reacted with alkylamine. such as propylamine or isopropylamine, to convert the pendent group into an alkylamide group. This reaction can also convert the polymerised residue derived from the third co-monomer (e.g. NAS) into an amide residue (e.g. acrylamido residue).

In another embodiment, the polymer conjugate may be prepared by polymerising a monomer composition comprising a plurality of different ethylenically unsaturated monomers, where at least one of the monomers comprises an agent conjugated thereto. Polymerisation of the monomer composition forms a linear, aliphatic, statistical copolymer backbone with one or more pendant agents. The agent-containing copolymer molecule may then be coupled to a binding moiety to form a polymer conjugate of the invention.

In another aspect, the present invention provides a process for preparing a biocompatible, hydrophilic polymer conjugate, the process comprising the steps of:
(a) polymerising a monomer composition comprising at least two different ethylenically unsaturated monomers and an ethylenically unsaturated monomer-agent conjugate under conditions of free radical polymerisation to thereby form a linear, aliphatic, statistical copolymer backbone having a pendant agent and a functional group at one or both ends of the copolymer backbone; and
(b) covalently reacting a binding moiety-containing molecule with a first functional group at a first end of the polymer backbone to conjugate the binding moiety to the first end.

In one embodiment, the process may further comprise the step of (c) covalently reacting an agent-containing molecule comprising an agent with a second functional group at a second end of the copolymer backbone to conjugate the agent to the second end.

In a third aspect, the present invention provides a process for preparing a biocompatible, hydrophilic polymer conjugate, the process comprising the steps of:
(a) polymerising a monomer composition comprising at least two different ethylenically unsaturated monomers and an ethylenically unsaturated monomer-agent conjugate under conditions of free radical polymerisation to thereby form a linear, aliphatic, statistical copolymer backbone having a pendant agent and a functional group at one or both ends of the copolymer backbone;
(b) covalently reacting a binding moiety-containing molecule with a first functional group at a first end of the polymer backbone to conjugate the binding moiety to the first end; and
optionally (c) covalently reacting an agent-containing molecule comprising an agent with a second functional group at a second end of the copolymer backbone to conjugate the agent to the second end.

As described herein, the functional group or groups situated at the end or ends of the copolymer backbone are considered to be terminal functional groups.

The ethylenically unsaturated monomers and the monomer-agent conjugate in the monomer composition of the third aspect preferably have different ethylenically unsaturated groups.

In some embodiments, the monomer composition of the above third aspect is polymerised under conditions of living free radical polymerisation, preferably reversible-addition-fragmentation-chain transfer (RAFT) polymerisation.

In one embodiment, the monomer composition comprises a monomer-agent conjugate of formula (III):

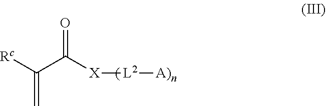

where:
   R^{c} is H or CH₃;
   X is selected from O or N;
   L² represents a linking moiety;
   A represents an agent; and
   n represents the number of (-L²-A) groups attached to X and is 1 or 2.

In a particular embodiment of the third aspect of the invention described herein, the monomer composition comprises:
(i) a first co-monomer selected from N-(2-hydroxypropyl)methacrylamide and N-(2-hydroxypropyl) acrylamide,
(ii) a second co-monomer selected from 2-hydroxyethyl acrylate, 2-methoxyethyl acrylate, 2-(diethylene glycol) ethyl acrylate, poly(ethylene glycol) acrylate, poly(ethylene glycol) methacrylate, poly(ethylene glycol) methyl ether acrylate, poly(ethylene glycol) methyl ether methacrylate, N-acryloylamido-ethoxyethanol, N,N-dimethylacrylamide, N,N-diethylacrylamide, N-(2-hydroxyethyl) acrylamide, N-(2-hydroxyethyl) methacrylamide, N-[Tris(hydroxymethyl)methyl]acrylamide, acrylamide, N-acryloylmorpholine, N-propyl acrylamide, N-isopropyl acrylamide, methacrylamide, di(ethylene glycol) methyl ether methacrylate, 2-hydroxyethyl methacrylate, 2-(dimethylamino) ethyl acrylate, 2-(diethylamino) ethyl acrylate, 3-(dimethylamino) propyl acrylate, (3-acrylamidopropyl) trimethylammonium chloride, 2-aminoethyl methacrylate hydrochloride, [3-(methacryloylamino)propyl]trimethylammonium chloride, 2-carboxyethyl acrylate, acrylic acid, N-carboxyethyl acrylamide, 2-acrylamido-2-methyl-1-propane sodium sulfonate, 3-sulfopropyl methacrylate potassium salt, 3-[[2-(acryloyloxy)ethyl]dimethyl-ammonio]propionate, 2-acryloyloxyethyl phosphorylcholine, [2-(acryloyloxy)ethyl]dimethyl-(3-sulfopropyl) ammonium hydroxide, N-(2-propynyl)-acrylamide, N-(3-azidopropyl)-acrylamide, and methacrylic acid, and
(iii) a third co-monomer which is a monomer-agent conjugate of formula (III):

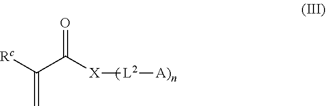

where:
   R^{c} is H or CH₃;
   X is selected from O or N;
   L² represents a linking moiety;
   A represents an agent; and
   n represents the number of (-L²-A) groups attached to X and is 1 or 2.

In one embodiment, the monomer-agent conjugate of formula (III) is acrylate monomer, where R^{c} is H and X is O.

In monomer-agent conjugates of formula (III), A may be a diagnostic or therapeutic agent.

In one embodiment, A is a therapeutic agent and L² is a biodegradable linking moiety, for example, an enzymatically cleavable linking moiety as described herein. A suitable enzymatically cleavable linking moiety may be valine-citrulline-para-aminobenzoic acid (Val-Cit-PABA), valine-alanine (Val-Ala), or phenylalanine-lysine (Phe-Lys).

The process of the third aspect also comprises the step of covalently reacting a binding moiety-containing molecule with a first terminal functional group at a first end of the polymer backbone to conjugate the binding moiety to the first end. Terminal functional groups suitable for conjugating a binding moiety either directly, or via a linker, are described herein.

In some embodiments, the binding moiety-containing molecule comprises a protein, preferably an antibody, an antibody fragment and an antigen binding fragment.

In a fourth aspect the present invention provides a method of alleviating, treating or preventing a disease or disorder in a subject comprising the step of administering to the subject, an effect amount of a polymer conjugate of any one of the embodiments described herein.

In particular embodiments, the polymer conjugate of the invention comprises an anti-neoplastic agent. In such embodiments, the invention may provide a method of treating cancer in a subject comprising the step of administering to the subject, an effect amount of a polymer conjugate of any one of the embodiments described herein comprising a anti-neoplastic agent.

The present invention also provides use of a polymer conjugate of any one of the embodiments described herein for targeted delivery of an agent to a desired cellular or tissue site. In particular embodiments, the agent is a diagnostic or therapeutic agent.

The present invention also provides a method of delivering an agent to a target cellular or tissue site in a subject, the method comprising the step of administering to the subject, an effective amount of a polymer conjugate of any one of the embodiments described herein.

In such methods, the binding moiety may be selected to target a desired cellular or tissue site to thereby facilitate site-specific delivery of the agent by the polymer conjugate. The agent may be a diagnostic agent or therapeutic agent, which can exert a desired effect at the target site.

### EXAMPLES

Synthesis of Polymer Backbones

Linear copolymers were made using RAFT polymerisation with initiators (4,4′-azobis(N,N,-cyanopentanoic acid, or V501) and RAFT agent (4-cyano-4-(phenylcarbonothioylthio) pentanoic acid) in either acetic acid-sodium acetate buffer, pH 5.2 (if making homopolymers) or ethanol (if making copolymers). A subset of polymers was selected that contained a range of homopolymers (as controls) and statistical copolymers (terpolymers) that are preferably biological compatible. The monomers chosen were: (N-(2-hydroxypropyl)methacrylamide (HPMA); N-acryloylmorpholine (NAM); N-isopropylacrylamide (NIPAM); polyethylene glycol methyl ether acrylate (PEGA); N-(2-propynyl)-acrylamide; and N-(2-hydroxypropyl)acrylamide (HPAm).

A number of model copolymers were prepared from a statistical mixture of two different co-monomers for initial bioconjugation, cytotoxicity and pharmacokinetic studies.

Polymer conjugates with a terpolymer backbone prepared with HPMA and NIPAM in a ratio of 1:1.4 (HPMA:NIPAM) and N-acryloylsuccinimide (NAS) were also prepared for the final drug-loading study. The NAS monomer was incorporated in the terpolymer backbone at a feed ratio of either 10% or 20%, relative to RAFT agent.

Each polymer prepared is composed of water soluble monomers and so the final polymers were all hydrophilic in nature. The method of polymerisation that was used was to combine all the monomers in the appropriate ratio at one time in a single reaction vessel and expose the mixture of monomers to the initiator and RAFT agent, thus leading to a statistical distribution of monomers along the growing polymer chain.

To form the polymers, a typical reaction solution was degassed by nitrogen bubbling for 1 hour and then stirred at 70° C. Monomer conversion was monitored by 1H NMR to calculate the number average molecular weight of the polymer. After 9 hours, the reaction was stopped by cooling to room temperature and opening to air. The control HPMA homopolymer, p(HPMA), was purified by precipitation from methanol into diethyl ether and then dialysis against H₂O. Copolymers and terpolymers were purified by precipitation from methanol into diethyl ether.

Attachment of Binding Moiety to Polymers

Installation of Linker at Either End of Polymer for Conjugation to a Binding Moiety

To attach a binding protein to a polymer it is necessary to install a linker such as a maleimide to the polymer for subsequent protein conjugation. Three different approaches for installing a maleimide containing linker were investigated, which are illustrated in Scheme 1:

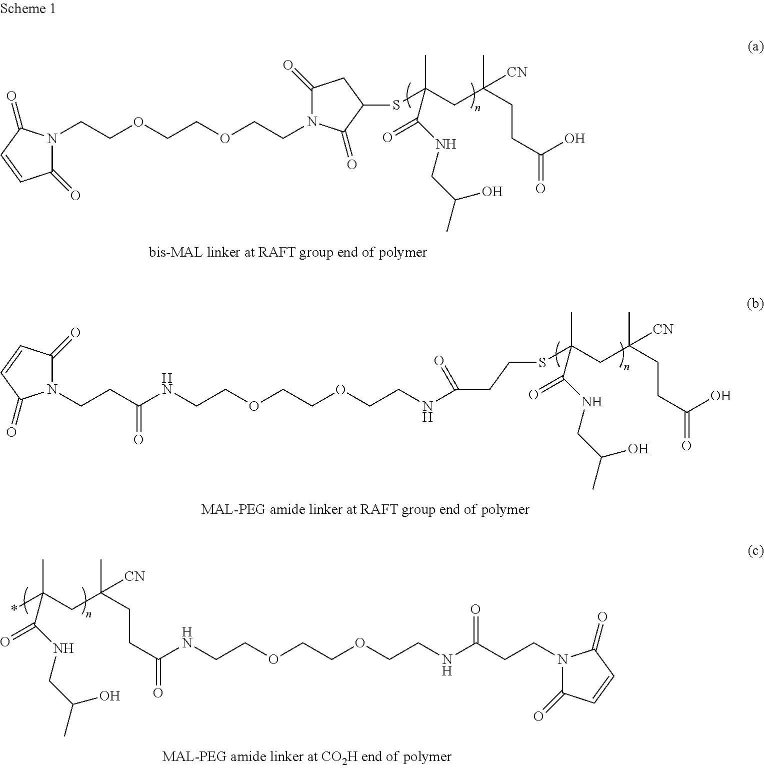

Option 1: Installation of Bis-Maleimide Linker at Thiol End of the Polymer

The use of a bifunctional bis-maleimide, (see Scheme 1(a)) was first developed. The RAFT end group on the polymer is removed by reaction with hexylamine, revealing a thiol.

Installation of the bis-malemide linker was achieved by reacting the thiol terminated polymer with a 20 equivalent excess of a PEG₂ bis-maleimide in the presence of 10 eq diisopropylethylamine in DMF. The maleimide modified polymer was then purified by dialysis to remove excess bis-maleimide.

Initial stability trials of polymer-Fab′ conjugates (analysis by size exclusion chromatography) showed that in buffer, (PBS), the original bis-maleimide linker bridging the RAFT polymer and antibody fragment can degrade with time and thus leads to the release of Fab′ which can be less than desirable in some clinical circumstances.

Option 2: Installation of Maleimide Linker at Thiol End of the Polymer

Another option to install a linker for conjugation to a binding protein is shown in Scheme 1(b). This option involves the use of an amide linkage, prepared by reaction of the polymer at a terminal thiol (SH group) which is revealed after RAFT end group removal, with a compound containing an acrylate or halo-alkyl that reacts with the SH, and which also has a carboxylic acid group. The carboxylic acid is then reacted with a PEG diamine, to couple one end of the PEG-diamine to the polymer, while the other end of the diamine remains free to react with a compound containing an NHS ester and a maleimide functional group.

Using this procedure, diisopropylethylamine (0.0486 mmol, 4 eq) and (1-cyano-2-ethoxy-2-oxoethylidenaminooxy)dimethylamino-morpholino-carbenium hexafluorophosphate (COMU, 0.0147 mmol, 1.1 eq) were added to a RAFT copolymer having a terminal thiol group in DMF (5 mL) at room temperature with stirring. After 2 mins, PEG₂-diamine (0.243 mmol, 20 eq) was added. After 4 hours, diisopropylethylamine (0.728 mmol, 60 eq) and N-succinimidyl 3-maleimidopropionate (maleimide-NHS) (0.728 mmol, 60 eq) were added with continued stirring. The solution was stirred at room temperature for another 2 hours, the product was purified by dialysis against water.

Option 3: Installation of Maleimide Linker at Carboxylic Acid End of the Polymer

Another method to install the maleimide to the CO₂H end of the polymer. In this option, a similar linker as that shown in Scheme 1(b) can be used at the carboxylic acid end of the linear copolymer. This option can involve the use of an amide linkage, which is prepared by reaction of the polymer having a terminal carboxylic acid (CO₂H) group at one end, with a PEG diamine, to couple one end of the PEG-diamine to the polymer, while the other end of the diamine remains free to react with a compound containing an NHS ester and a maleimide functional group. The RAFT end group at the other end of the polymer and the resulting thiol can also be removed completely (Scheme 1(c)).

Installation of the malemide linker was achieved by completely removing the RAFT end group by treatment with hypophosphite, then the carboxylic acid at the R-end of the polymer was activated by COMU and reacted with an excess of PEG2-diamine. After purification by dialysis or precipitation the amine-PEGylated polymer end group was reacted with N-succinimidyl 3-maleimidopropionate to install a maleimide at the carboxylic acid-end of the polymer.

Similar linker strategies as that above used for attaching a binding moeity to the polymer can also be employed to attach an agent, such as a cytotoxic agent, an imaging agent, dye or any small molecule of therapeutic or biological relevance, to an end of the polymer as well.

Conjugation of Binding Moiety (Protein Fab′) to a Terminal End of Polymer

By adding a binding moiety such as a targeting antibody fragment to the end of the copolymer, the resulting conjugate will give rise to an increase in tumour targeting by receptor-mediated delivery. For efficacy, the copolymer needs to be non-toxic and non-immunogenic and the molecular weight needs to be high enough to guarantee a relevant increase in circulating half-life to allow for accumulation, but then be capable of renal clearance after drug delivery. It is also important that the copolymer-protein end linker is also physiologically stable throughout the treatment period.

The protein (i.e. binding moiety) could be attached/conjugated to the copolymer before or after attachment of the agent.

In experiments illustrated below, protein conjugation occurs before the attachment of a clinically relevant drug.

To demonstrate attachment of a protein at the end of the copolymer, a well-defined clinically relevant antibody, an anti-EGFR antibody, mAb 528, which binds to the EGFR blocking ligand binding and receptor activation in a manner identical to that of Cetuximab, was selected. The mAb 528 was purified from ATCC HB8509 conditioned media by affinity purification (AbCapcher, Cosmobio) and cleaved with pepsin to produce the Fab′2 fragment, which was purified by gel filtration chromatography. Reduction trials with mercaptoethanol, DTT and TCEP demonstrated the 528 Fab′ interchain disulphide was particularly prone to reduction. The best conditions were with 1.5 mol TCEP per mol Fab′2 fragment which yielded approximately 50% Fab′ with no detectable reduction of the interchain disulphide bond, and the Fab′ fragment was separated from the non-reduced Fab′2 by gel filtration chromatography. The gel filtration isolation of the Fab′ fragment prior to conjugation was necessary in some cases, since the conjugation reaction product often eluted from the gel filtration column at a volume similar to that of Fab′2. Even with gel filtration isolation of Fab′ prior to conjugation, some Fab′2 was reformed under the conjugation conditions, and this material was poorly separated from the conjugate by gel filtration. This requirement for isolation of the Fab′ fragment, and the gradual oxidation of the Fab′ to Fab′2 on storage meant there was an upper limit to the amount of Fab′ that could be prepared for a given conjugation experiment (about 50 mg Fab′ using a GE Healthcare Superdex S200 2660 column).

To generate larger amounts of material and to improve the separation of the Fab′2 from the Fab′-conjugate, conjugation directly to the Fab′ in the reduction mixture without prior separation was carried out. This was achieved with cation exchange chromatography, the Fab′-conjugate eluting earliest from the column and the Fab′2 retained longest.

Various model polymers with different maleimide-containing linkers as shown in Scheme 2 were prepared. The model polymers included a bis-maleimide linker installed onto the terminal thiol of a RAFT polymer (Scheme 2(A)), a comparative PEG polymer (20 kDa) having a maleimide installed on the terminus of the polymer (Scheme 2(B)), a maleimide installed to terminal thiol of a RAFT copolymer via PEG-amide linker (Scheme 2(C)), and a maleimide installed onto a terminal carboxylic acid of a RAFT copolymer via a PEG-amide linker (Scheme 2(D)).

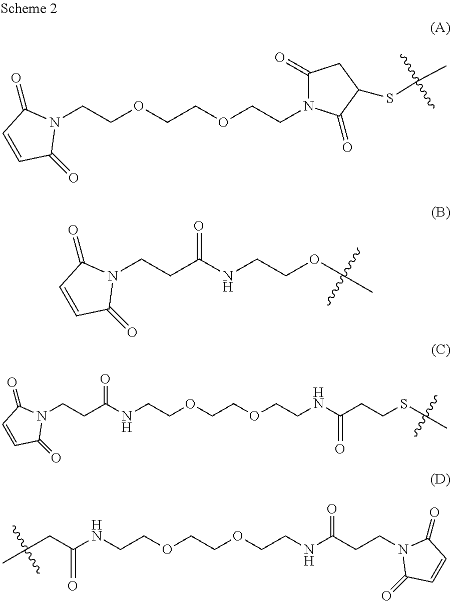 [SMILES: *SC1CC(=O)N(CCOCCOCCN2C(=O)C=CC2=O)C1=O ; *OCCNC(=O)CCN1C(=O)C=CC1=O ; *SCCC(=O)NCCOCCOCCNC(=O)CCN1C(=O)C=CC1=O ; *CC(=O)NCCOCCOCCNC(=O)CCN1C(=O)C=CC1=O]

Protocols for installing the maleimide containing linkers at either the thiol end or carboxylic end of the model RAFT polymers are described above.

For protein-polymer bioconjugation studies, model RAFT polymers with a maleimide-containing linker at the carboxylic acid end of the polymer were prepared. These are detailed in Table 1. Additionally, a commercially available PEG-MAL was also used to prepare a comparative control PEG-Fab′ polymer (Table 1).

TABLE 1
Polymers with maleimide-containing linkers for protein conjugation study

| | | | | Target |
|---|---|---|---|---|
| | | | | maleimide at |
| | | | Location of | the end of |
| | | PDI | maleimide | polymer chain |
| | MW | (DMAC- | linker on | (% by ¹H |
| Polymer | (¹H NMR) | GPC) | polymer | NMR) |
| p(HPMA) | 32740 | 1.12 | CO₂H end | ~90 |
| p(HPMA- | 35547 | 1.17 | CO₂H end | 65 |
| NAM) (1:1) | | | | |
| p(HPMA- | 27676 | 1.2 | CO₂H end | 75 |
| NIPAM) (1.4:1) | | | | |
| p(HPMA-PEG) | 36199 | 1.25 | CO₂H end | 90 |
| (1:1) | | | | |
| p(NAM) | 35148 | 1.18 | CO₂H end | 88 |
| p(HPMA- | 25439 | 1.23 | CO₂H end | 72 |
| NIPAM) (1:1) | | | | |
| p(HPAm) | 25466 | 1.17 | CO₂H end | 74 |
| PEG | 20000 | <1.1 | — | 60 |

p(HPMA) = poly(N-(2-hydroxypropyl)methacrylamide;
p(NAM) = poly(N-acryloylmorpholine);
NIPAM = N-isopropylacrylamide;
p(HPAm) = poly(N-(2-hydroxypropyl)acrylamide;
PEG = poly(ethylene glycol).

Protein Fab′ Conjugation with Polymers

The maleimide-containing model RAFT polymers and the comparative PEG-MAL were conjugated with Fab′-SH via the maleimide functional group. Fab′ conjugation with the maleimide-containing polymers was performed by treating 1 equivalent of Fab′ with from 0.5-2 equivalents of maleimide-containing polymer for up to 40 h at 4° C., with the pH varied from pH 6-8.

The highest yield of Fab′-conjugate was with 1.5-2 equivalents of the polymer, but these conditions produced some higher molecular weight doubly-conjugated Fab′ (e.g., eluting at 10 mL so higher amounts of copolymer were not examined. The reaction was found to be complete after 1 h, and pH 7-7.5 was found to give the highest yield of product.

The model Fab′-RAFT polymers were found to be less stable than the comparative Fab′-PEG polymer, with breakdown of the Fab′-RAFT polymer conjugate observed even after 5 days at 4° C. Both the RAFT and PEG polymers had a maleimide installed at one end of the polymers for reaction to a free thiol on the Fab′.

Antibody-drug conjugates using maleimide chemistry are used clinically but can have potential problems with instability. The precise local environment of the thiol to which the maleimide is conjugated has been shown to greatly affect the stability of the maleimide linkage. Since the maleimide on the PEG was more stable than the linkage on the RAFT polymer, it would seem probable the difference in stability is due to the maleimide-polymer link, not the maleimide-protein link.

Conjugation of Protein Fab′ to Carboxylic Acid Functional Group at End of Model Copolymer and Assessment of Hydrodynamic Volume

A series of model tritiated RAFT polymers of different compositions (Table 2) were prepared on a large scale, with the maleimide installed at the carboxylic acid end of the copolymers as previously described above, and these were conjugated to the Fab′-SH (Scheme 3). The composition and molecular weights of the polymers were chosen to give approximately similar gel filtration elution volumes in PBS, which gives an indication of the hydrodynamic radius of the Fab′-conjugate and hence the clearance volume.

To Fab′2 (173 mg, 1.7 mmol) in PBS reduced with 1.5 equivalents of TCEP for 2 h at 25° C. was added 1.5 equivalents of maleimide-containing model RAFT polymer or comparative PEG polymer and the mixture allowed to react for 1 h at 4° C. with mixing.

The mixture was diluted 10-fold with 10 mM MES pH 6 and applied to two 5 mL HiTrap SP HP columns (GE Healthcare) connected in series. The unbound material was washed and the protein eluted with a linear gradient from 0-1 M NaCl in the same buffer. The early eluting material was pooled and subjected to gel filtration on a Superdex S 200 2660 column. The pooled material was concentrated and found to be greater than 95% pure as judged by the gel filtration profile (Superdex S200 1030 column). The conjugation yields for the RAFT-derived model copolymers were comparable to that for PEG (Table 2).

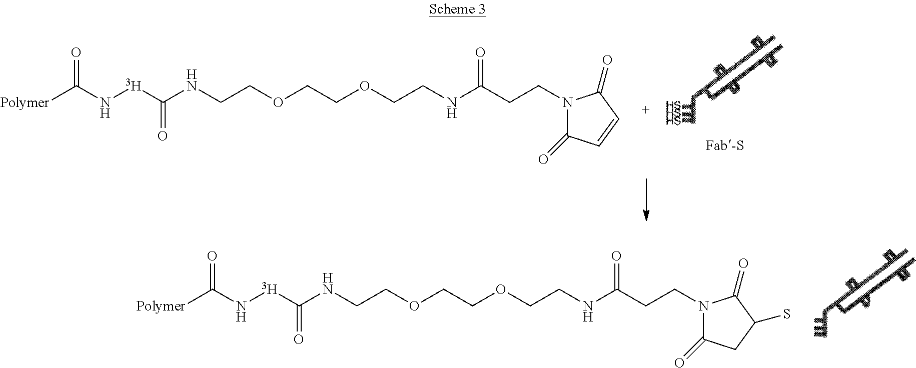

TABLE 2
Yield from large scale synthesis of tritiated model Fab′-conjugated copolymers
before adding in the drug.

| | | | Fab-conj | Fab′-conj | Yield | | | |
|---|---|---|---|---|---|---|---|---|
| | | | post SP | post GF | (protein) | | | Specific |
| | | | (mg of | (mg of | (% from | | Endotoxin | activity |
| | Polymer | MW | protein) | protein) | Fab′2) | Purity | units/mg | (kBq/mg) |
| A | p(HPMA) | 32740 | 38 | 34.2 | 19.8 | 95 | 0.107 | 60.77 |
| B | p(HPMA- | 35547 | 62 | 45.5 | 26.3 | 100 | 0.058 | 104.72 |
| | NAM) | | | | | | | |
| C | p(HPMA- | 27676 | 54 | 46.8 | 27.1 | 98 | 0.236 | 82.77 |
| | NIPAM) | | | | | | | |
| | ratio 1 | | | | | | | |
| D | p(HPMA- | 36199 | 50.6 | 45.5 | 26.3 | 98 | 0.149 | 85.07 |
| | PEGA) | | | | | | | |
| E | p(NAM) | 35148 | 40.4 | 26.8 | 15.5 | 100 | 0.101 | 83.43 |
| F | p(HPMA- | 25439 | 43 | 29.6 | 17.1 | 99 | 0.148 | 110.34 |
| | NIPAM) | | | | | | | |
| | ratio 2 | | | | | | | |
| G | p(HPAm) | 25466 | 39.9 | 27.7 | 16.0 | 100 | 2.999 | 75.68 |
| H | PEG | 20000 | 42 | 33.7 | 19.5 | 100 | <0.088 | 97.17 |

Ratio 1 = 1:1 molar ratio of HPMA:NIPAM
Ratio 2 = 1.4:1 molar ratio of HPMA:NIPAM

In the above table, samples A-G are model RAFT derived polymers of differing compositions derived from either one monomer or two co-monomers, while H is maleimide-PEG 20 kDa. The purity was estimated by analytical gel filtration. Endotoxin was assayed using the Endosafe PTS system (Charles River Laboratories).

The gel filtration elution volume provides an indication of hydrodynamic volume, which is important for understanding the comparison between polymer compositions and the effect that the polymers have on pharmacokinetics. Polymers selected for gel filtration studies are matched for ‘size’ in water (i.e. plasma) rather than size as per determined by ¹H NMR. This allows the polymers to be more realistically matched for comparison in terms of size in the circulation. Observed differences in pharmacokinetics are therefore due to differences in polymer composition rather than simply the size of the polymer.

In Vitro Studies of a Model Fab′-RAFT Polymer Conjugate (without an Agent Component)

In Vitro Studies of Model Fab′-RAFT Polymer and Comparative Fab′-PEG Conjugates (Fab′ Activity and Cell Toxicity of Polymers within Conjugates).

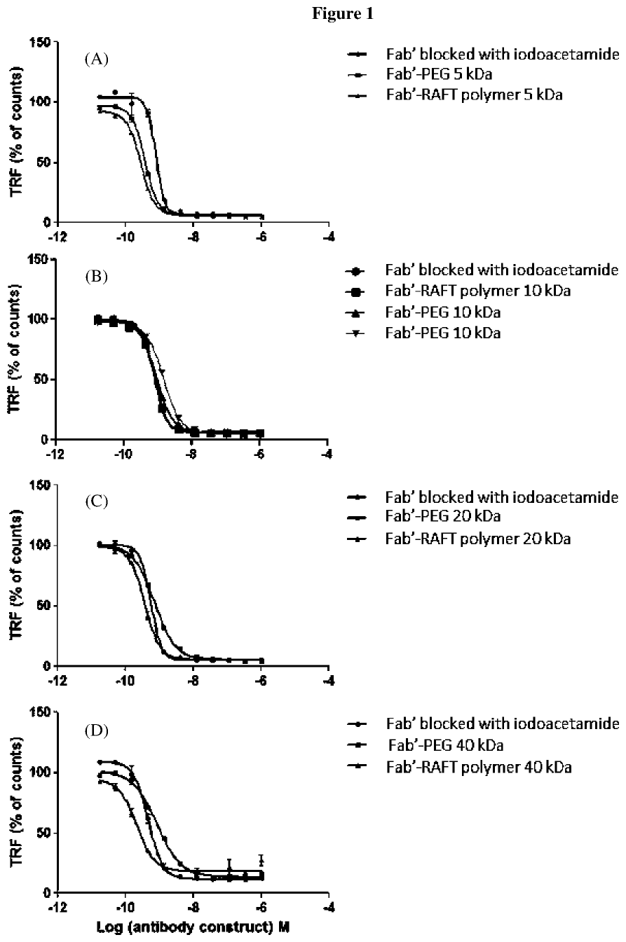

The mechanism of action of mAb 528 involves direct binding to the EGFR, thereby blocking the binding of its cognate EGF family of ligands. In an in vitro competition-based binding assay the ability of conjugated Fab′ fragments to compete with Europium-labelled EGF in binding to immobilised EGFR was assessed for a number of model Fab′-RAFT polymers (Fab′-p(HPMA)) of different molecular weight and size-matched comparative Fab′-PEG conjugates. The results obtained from four dose-response competition binding assays are presented as examples, for model RAFT-derived polymers with MW of 5, 10, 20 and 40 kDa (FIG. 1). Each of the different MW model Fab′-polymer conjugates bound to EGFR with affinity similar to that of the both unconjugated Fab′, and the respective comparative Fab′-PEG conjugate. Analysis of these data sets demonstrated clearly that conjugation of various molecular weight model RAFT polymers and PEG polymers to the Fab′ did not result in steric interference with antigen recognition.

Upon ligand binding, EGFR on the cell surface dimerises, inducing a conformational change that results in the activation of the tyrosine kinase activity of the receptor and subsequent downstream signalling events. By blocking binding of the cognate ligand, mAb 528 and fragments thereof, can prevent dimerisation of this receptor and the subsequent phosphorylation of the receptor and other substrates.

The efficiency of the model Fab′-RAFT polymer conjugates in binding to purified EGF receptor was assessed via a cellular signalling system, using human ACHN kidney carcinoma cells, which express high levels of EGFR on their cell surface. A quantitative estimate of receptor phosphorylation can be obtained following ligand stimulation of cells by solubilising the cell monolayer, and capturing the EGFR in antibody-coated wells. The level of tyrosine phosphorylation can be measured by incubating with Europium-labelled, anti-phosphotyrosine antibody.

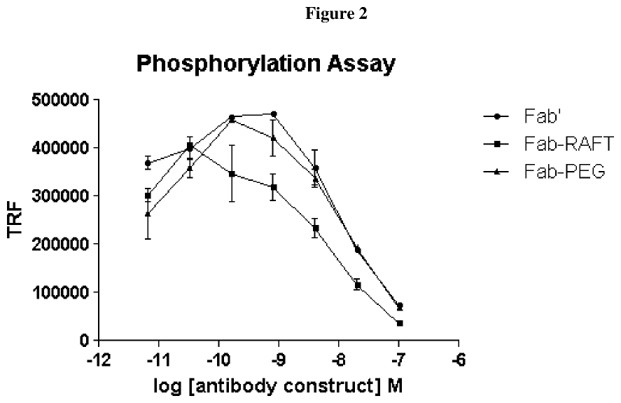

As illustrated in FIG. 2 both the free Fab′ and comparative Fab′-PEG conjugates effectively inhibited ligand-induced signalling, as indicated by a reduction in time-resolved fluorescence TRF: representing tyrosine phosphoryated EGFR) in a manner that was superimposable. The model Fab′-RAFT polymer conjugate was also effective at inhibiting receptor activation, perhaps more so than the Fab′-PEG conjugate. Again, these results confirm that conjugation of polymers prepared using RAFT to mAb 528 Fab′ fragments did not compromise antigen recognition and subsequent downstream phosphorylation of the receptor complex.

The potential toxicity of p(HPMA) RAFT polymer in comparison to a PEG polymer was assessed in a cell based toxicity assay using mouse L929 fibroblasts. Briefly, cells were plated out in 96 well plates and allowed to attach overnight. The following day the cells were exposed to different concentrations of polymer in growth medium supplemented with foetal bovine serum. The effect of the polymers on cell growth as a measure of toxicity was assayed 20-24 hours later using a colorimetric test of cell viability. There was very little difference in the toxicity profile between the RAFT-derived and PEG polymers observed with apparent cell death being observed only at the higher concentrations of polymer (>1 mg/mL).

In Vivo Studies of Model RAFT Copolymers

Preparation of Tritium Labelled Model RAFT Polymers with Terminal Maleimide Functional Group

Labelling with tritium (³H) and monitoring the radioisotope by scintillation counting, is a standard method used for monitoring a compound in vivo, for example in an ADMET (absorption, distribution, metabolism, excretion, toxicity) study. A ³H label was introduced to model RAFT polymers by installation of a 3H radiolabelled glycine residue at the R-group, or carboxylic acid end, of the polymer (Scheme 4). This was done using peptide coupling techniques, followed by further modification to the CO₂H of the ³H glycine with a diamine (PEG₂-diamine), then reaction of a free amine from the diamine with N-succinimidyl 3-maleimidopropionate to give rise to radiolabelled polymers containing a terminal reactive functional group (maleimide) at the carboxylic acid end of the polymer, which is suitable for conjugation to the antibody.

As a control and a comparison for the model polymers, a poly(ethylene glycol) (PEG) polymer, that has a similar hydrodynamic volume was used. The PEG was similarly radiolabelled by reaction with ³H-glycine and a terminal reactive maleimide was installed for conjugation to the Fab′-SH. Maleimide functionalisation was achieved by reacting the glycine residue with an excess of PEG₂-diamine (with COMU coupling agent), after dialysis to remove excess diamine, the terminal amine was then reacted with this N-succinimidyl 3-maleimidopropionate to provide a maleimide functionalised PEG polymer.

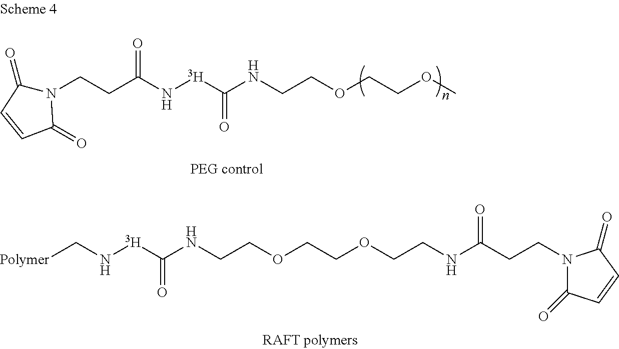

### Experimental

(a) RAFT Polymers

At room temperature, RAFT polymer (0.024 mmol) was weighed and dissolved in DMF (8 ml). Diisopropylethylamine (DIEA, 0.1 mmol) and COMU (0.0264 mmol) were added to the polymer solution. After 3 minutes, tritiated glycine (0.024 mmol) in deionised water (0.1 ml) was added to the reaction solution. The reaction solution was then left at room temperature overnight. 0.1 N HCl (1 ml) was added to acidify the reaction solution. The tritium labelled polymer was purified by dialysis against deionised water and then freeze dried.

The purified polymer was dissolved in DMF (8 ml). DIEA (0.1 mmol), NHS (0.048 mmol) and COMU (0.048 mmol) were added to the polymer solution. The reaction solution was left overnight. 2,2′-(Ethylenedioxy)bis(ethylamine) (EDEA, 0.48 mmol) and DIEA (0.96 mmol) were added to the reaction solution. After 4 hours, N-succinimidyl 3-maleimidopropionate (1.44 mmol) was added to the reaction solution. After another 1 hour, acetic acid (2.16 mmol) was added to acidify the reaction solution. The final product was purified by dialysis against deionised water and then freeze dried.

(b) PEG Control

N-succinimidyl 3-maleimidopropionate (0.03 mmol) and DIEA (0.048 mmol) was dissolved in DMF (3 ml). Tritiated glycine (0.024 mmol) was added and then left overnight at room temperature. COMU (0.029 mmol) and DIEA (0.048 mmol) were added to the reaction solution. After 3 minutes, PEG-NH₂ (MW ˜20000, 0.024 mmol) and DIEA (0.024 mmol) were added to the reaction solution. After another 2 hours, acetic acid (0.24 mmol) was added to acidify the reaction solution. The final product was purified by dialysis against deionised water and then removed the water by rotavapor.

The synthesised polymers are shown in Table 3.

Results:

TABLE 3
³H labelled model and comparative polymers with maleimide
end group installed.

| | | | Target | |
|---|---|---|---|---|
| | | | maleimide at | |
| | | | the end of | |
| | MW | | polymer chain | Polymer |
| Polymer | (1H | PDI | (% by 1H | total |
| composition | NMR) | (DMAC-GPC) | NMR) | mass (g) |
| p(HPMA) | 32740 | 1.12 | ~90 | 0.7698 |
| p(HPMA- | 35547 | 1.17 | 65 | 0.8153 |
| NAM) (1:1) | | | | |
| p(HPMA- | 27676 | 1.2 | 75 | 0.6406 |
| NIPAM) (1.4:1) | | | | |
| p(HPMA-PEG) | 36199 | 1.25 | 90 | 0.8224 |
| (1:1) | | | | |
| p(NAM) | 35148 | 1.18 | 88 | 0.8001 |
| p(HPMA- | 25439 | 1.23 | 72 | 0.5857 |
| NIPAM) (1:1) | | | | |
| p(HPAm) | 25466 | 1.17 | 74 | 0.4580 |
| PEG | 20000 | <1.1 | 60 | 0.4684 |

p(HPMA) = poly(N-(2-hydroxypropyl)methacrylamide;
p(NAM) = poly(N-acryloylmorpholine);
NIPAM = N-isopropylacrylamide;
p(HPAm) = poly(N-(2-hydroxypropyl)acrylamide;
PEG = poly(ethylene glycol).

In Vivo Studies Showing ADMET of Model RAFT Copolymers Alone (not Conjugated to Fab′):

A selection of tritium labelled model RAFT polymers were prepared as above and subjected to ADMET profiling. These model polymers are shown in Table 4.

TABLE 4

| | | | | Specific |
|---|---|---|---|---|
| | Reference in | | | radioactivity |
| Polymer | this study | MW (kDa) | PDI | (μCi/mg) |
| pHPMA | 6A | 27 | 1.18 | 0.314 |
| p(HPMA-PEG) | 8 | 13 | 1.26 | 0.646 |
| p(HPMA-NAM) | 13 | 35 | 1.29 | 0.237 |
| p(HPMA-NIPAM) | 16 | 18 | 1.25 | 0.303 |

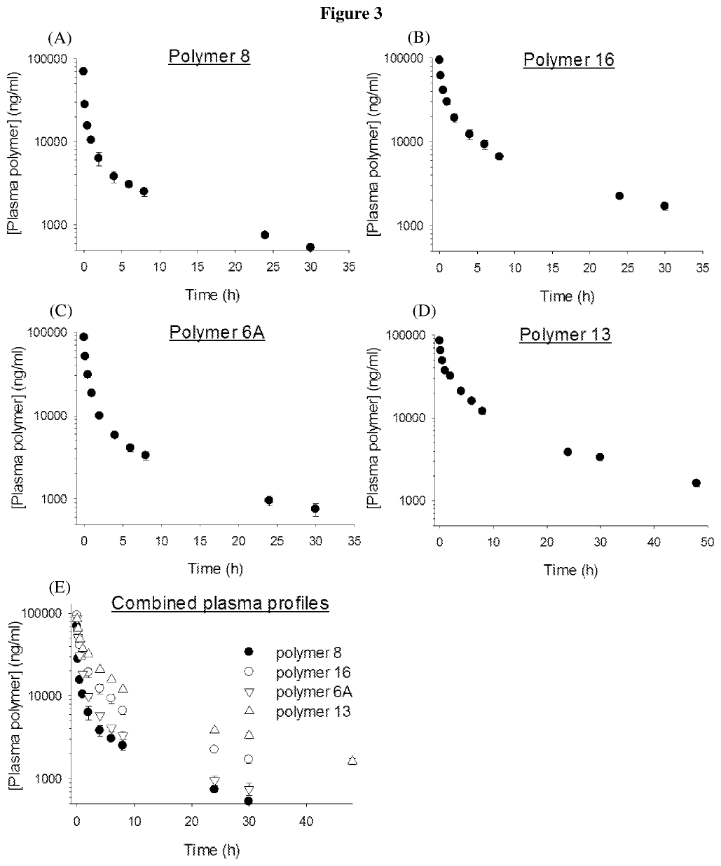

The polymers were administered to Sprague-Dawley rats (5 mg/kg) and the radioactivity remaining in the blood determined by scintillation counting. The concentration of polymers remaining in the blood decreased over time with the highest MW polymer, polymer 13 (p(HPMA-NAM), 35 kDa), having the slowest clearance rate from the plasma (3.1±0.1 mL/h), while the smallest polymer, polymer 8 (p(HPMA-PEG), 13 kDa) was cleared the fastest (15.7±1.2 mL/h). Interestingly, polymer 16 (p(HPMA-NIPAM)) with a MW of only 18 kDa was cleared more slowly (5.5±0.5 mL/h) than the much larger polymer 6A (pHPMA, 27 kDa), (9.1±0.2 mL/h). See FIG. 3.

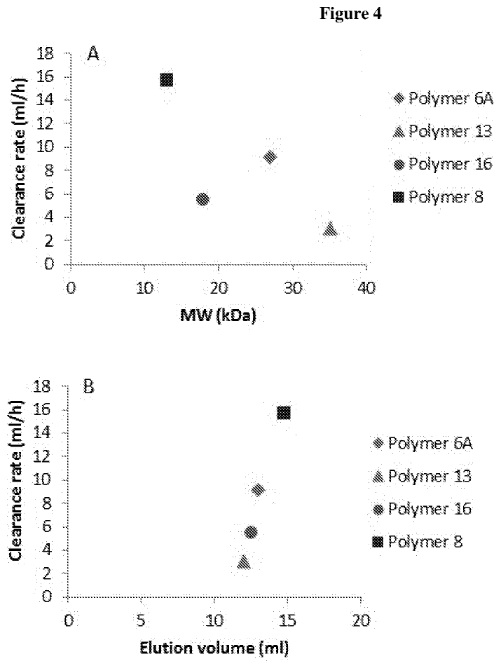

It is worth noting that while the clearance time of the polymers was only loosely dependent on the molecular weight of the polymers, there was a much stronger relationship between the clearance time of the polymers and their elution volume from the size exclusion column (FIG. 4). This is perhaps not surprising as the elution volume in size exclusion is related to the hydrodynamic radius of the polymer, and it is the hydrodynamic radius rather than the molecular weight which would be expected to be more predictive of the extent of renal filtration and excretion.

This study showed that Polymer 13 had the longest retention while polymer 8 had the shortest retention, possibly just due to size. However, it was also found that polymer 6A had a shorter retention than expected for its size, and polymer 16 had a longer retention than expected for its size.

In Vivo Studies Showing ADMET of Model Fab′-RAFT Polymer and Comparative Fab′-PEG Conjugates:

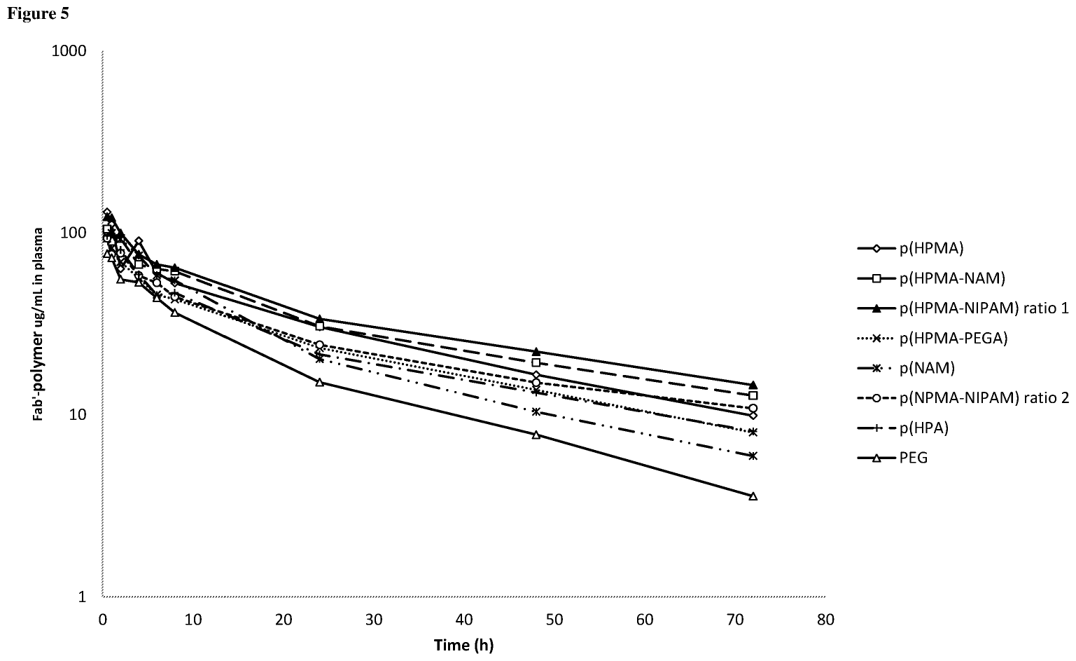

Evaluation of the uptake and distribution of eight model Fab′-conjugated polymer variants of Table 2 was followed after a single intravenous dose administration in rats. The study was performed on female Sprague-Dawley rats randomly divided into 8 groups, with three sub-groups in each group (4 animals per subgroup). The conjugates were administered by a single IV injection at the rate of 5 mg of Fab′-conjugate per kg. The animals were bled over several time intervals. As radioactively labelled polymers were used, these samples revealed (by scintillation counting, Perkin Elmer) the concentration of Fab′-polymer conjugate in plasma. The concentration of the Fab′-polymer conjugate in plasma decreases rapidly over the first 8 hours, as the material is distributed throughout the organs of the rat (alpha phase) then is eliminated with the expected first-order kinetics (beta phase). From the 24-72 h data points, the rate of elimination (k) was determined (FIG. 5). The elimination half-life (T½β) was calculated as the ln 2/k (Table 5).

TABLE 5

| | | | | Apparent | | |
|---|---|---|---|---|---|---|
| | | MW | | MW | | |
| | | (Fab′- | | (conjugate, | Elimination | |
| | MW | polymer | Elution | gel | rate | |
| | (polymer) | conjugate) | volume | filtration) | constant | |
| Polymer | (Da) | (Da) | (mL) | (Da) | (h-1) | T½β |
| p(HPMA) | 32740 | 82270 | 12.64 | 218000 | 0.02341 | 29.6 |
| p(HPMA- | 35547 | 85077 | 11.95 | 301000 | 0.01826 | 38.0 |
| NAM) (1:1) | | | | | | |
| p(HPMA- | 27676 | 77206 | 11.97 | 299000 | 0.01746 | 39.7 |
| NIPAM) | | | | | | |
| (1.4:1) | | | | | | |
| p(HPMA- | 36199 | 85729 | 12.56 | 226000 | 0.02227 | 31.1 |
| PEGA) | | | | | | |
| (1:1) | | | | | | |
| p(NAM) | 35148 | 84678 | 12.12 | 278000 | 0.02551 | 27.2 |
| p(HPMA- | 25439 | 74969 | 11.96 | 300000 | 0.01672 | 41.5 |
| NIPAM) | | | | | | |
| (1:1) | | | | | | |
| p(HPAm) | 25466 | 74996 | 12.42 | 242000 | 0.02026 | 34.2 |
| PEG | 20000 | 69530 | 11.56 | 362000 | 0.03003 | 23.1 |

All the model Fab′-polymer conjugates were retained in the plasma for longer than would be expected for an unmodified Fab′, with half-lives comparable to those of comparative Fab′-PEG conjugates of 75-100 kDa (Table 6). The model polymers were selected to have similar hydrodynamic radii (as estimated from gel filtration chromatography of the polymers in aqueous buffer), and the elution volumes of the model Fab′-polymer conjugates vary from 11.6-12.6 mL, corresponding to an apparent MW of the conjugate of 220 kDa (Fab′-p(HPMA)) to 360 kDa (Fab′-PEG). The elimination half-life is not directly related to either MW (as judged by NMR of the polymer) or the apparent MW (as estimated by gel filtration) since the model Fab′-polymer conjugates with the longest T½β, p(HPMA-NIPAM) (ratios 1 and 2) and p(HPMA-NAM) had MWs of 75-85 kDa and apparent MWs by gel filtration of about 300 kDa. The comparative Fab′-PEG had a much larger apparent MW by gel filtration of 362 kDa (although an actual MW of only 70 kDa) yet a significantly shorter half-life of only 23 h.

TABLE 6

| | Molecular weight | |
|---|---|---|
| Construct | (kDa) | T1/2β (h) |
| Fab′ | 50 | 22.7 |
| Fab′-PEG 1 × 25 kDa | 75 | 30.5 |
| Fab′-PEG 1 × 40 kDa | 90 | 45.8 |
| Fab′-PEG 2 × 25 kDa | 100 | 49.1 |

The amount of Fab′-conjugate excreted in the urine for a 6 h time period was determined for each of the model Fab′-polymer conjugates. The amount of Fab′-conjugate in urine for the 48-56 h time period was estimated to account for from 50-100% of the material eliminated from the plasma over the same time period.

Synthesis and Assessment of Model Agent (Dye)—Copolymer-Fab′ Conjugates.

Dye (Texas Red) labelled model polymer conjugates were prepared by installing the dye at different locations on a model p(HPMA) backbone or a model p(HPMA-co-N-2-propynyl acrylamide) backbone. Subsequent conjugation of the dye-labelled model polymers to Fab′ then followed.

The conjugation of the dye also helped ascertain the stability of the conjugate. Stability experiments were conducted by incubating the dye conjugates in PBS and rat serum, and the breakdown of the conjugate was detected by co-analysing protein (Amax 280 nm) and dye (Amax 589 nm) absorbance maxima in size exclusion chromatography (see in vitro results section below).

In one experiment, a dye is attached to an end of linear copolymer backbone. In this experiment, a model poly(hydroxypropylmethacrylamide (p(HPMA)) polymer was formed by RAFT polymerisation following the general protocol described above. The molecular weight of the polymer was between 20-30 kDa. After p(HPMA) formation, the CO₂H end of the polymer was reacted with an amine-functionalised Alexa Fluor 488 dye. After purification by dialysis or precipitation, the RAFT end group was removed by treatment with an excess of hexylamine to reveal a terminal thiol at the end of the polymer chain.

The thiol was then reacted with a PEG₂-bismaleimide in 20 fold excess, which after dialysis, yielded a MAL-functionalised dye-loaded polymer (Scheme 6(a)).

In another experiment, a dye is attached to and pendant from the linear copolymer backbone. In this experiment, a model p(HPMA-co-N-2-propynyl acrylamide) backbone was prepared by RAFT polymerisation of HPMA (1 eq.) and N-2-propynyl acrylamide (10 eq.) following the general protocol described above. After formation of the copolymer, the RAFT end group was removed completely by treatment with hypophosphite, and the CO₂H end of the polymer reacted under peptide coupling conditions, using COMU as a coupling agent, with a PEG₂-diamine. The amine functionalised copolymer was then purified by dialysis or precipitation. Subsequently, the amine terminated polymer was reacted with N-succinimidyl 3-maleimidopropionate to install a maleimide linker (MAL) at the end of the polymer. The dye (azido Texas Red) was conjugated to pendant alkyne functional groups in the copolymer, which are provided by polymerised residues derived from the N-2-propynyl acrylamide co-monomer. Conjugation of the dye to the pendant alkyne groups proceeded under click chemistry conditions (Scheme 6(b)).

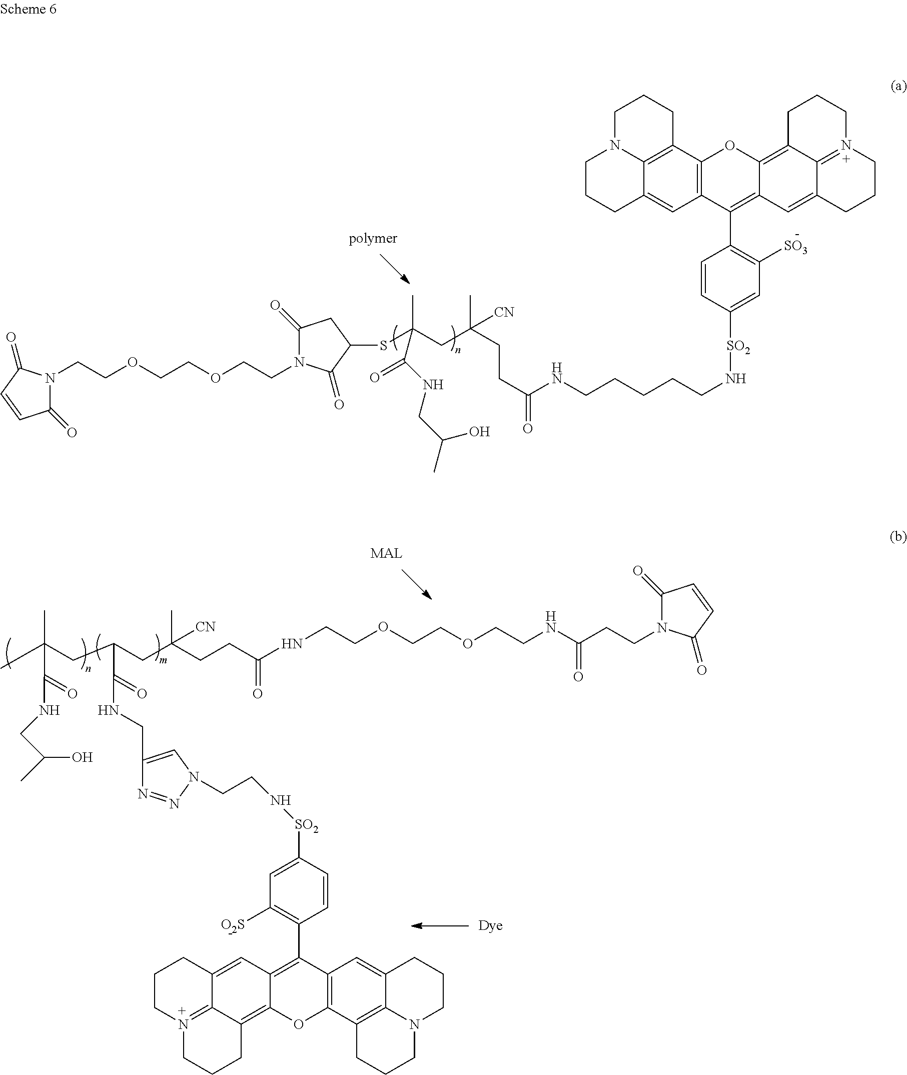

Dye-labelled, MAL-installed comparative and model polymers with different MAL linker types and with the MAL linker located at either end of the polymer backbone were subsequently conjugated to an antibody fragment. In order to assess physiologically relevant stability the conjugates were purified first by gel filtration chromatography (GFC) prior to incubation with PBS or serum.

Results showed that polymer conjugates using a bis-maleimide linker to conjugate the antibody fragment were less stable than the alternative MAL linkers. Furthermore, when the alternative MAL linkers were used, it did not matter to which end of the polymer the alternative linkers were attached. The dye conjugated polymers could then be reacted with protein (i.e. binding moiety) at the MAL ends of the polymer.

Synthesis of Agent (Drug)—Terpolymer—Fab′ Conjugates.

To investigate biological targeted delivery of a therapeutic drug (as opposed to the dye scenario), the cytotoxic drug monomethyl auristatin E (MMAE), which is a synthetic small molecule, antineoplastic agent, was selected for attachment to a RAFT copolymer, which is a terpolymer. This drug/agent was attached to the polymer via enzymatically cleavable linker chemistry (ValCitPABA), whereby free, unmodified MMAE is released upon selective cleavage of the dipeptide linker which is attacked by specific enzymes at the tumour site. The copolymer of choice for this targeted, drug-loaded study comprised N-(2-hydroxypropyl)methacrylamide, (HPMA) as a first monomer, which was chosen for its biologically applicability. The second monomer was N-isopropylacrylamide (NIPAM). From ADMET studies it was found that a copolymer having HPMA and NIPAM residues had the longest retention while a copolymer having HPMA and PEG had the shortest retention. It was also found that p(HPMA) homopolymer had a shorter retention than expected for its size, but that p(HPMA-NIPAM) had a longer circulating retention than expected for its size. Given these results, a terpolymer formed with HPMA and NIPAM as first and second monomers, were chosen for the drug loading study.

A third co-monomer for carrying the agent (drug) was introduced also into the copolymer.

Linker molecules for conjugating the drug to the copolymer were prepared by different synthesis methods, as shown below:

Method 1.

Preparation of Boc-PEG-Val-Cit-OH 1 using solid-phase peptide synthesis protocols (Scheme 7).

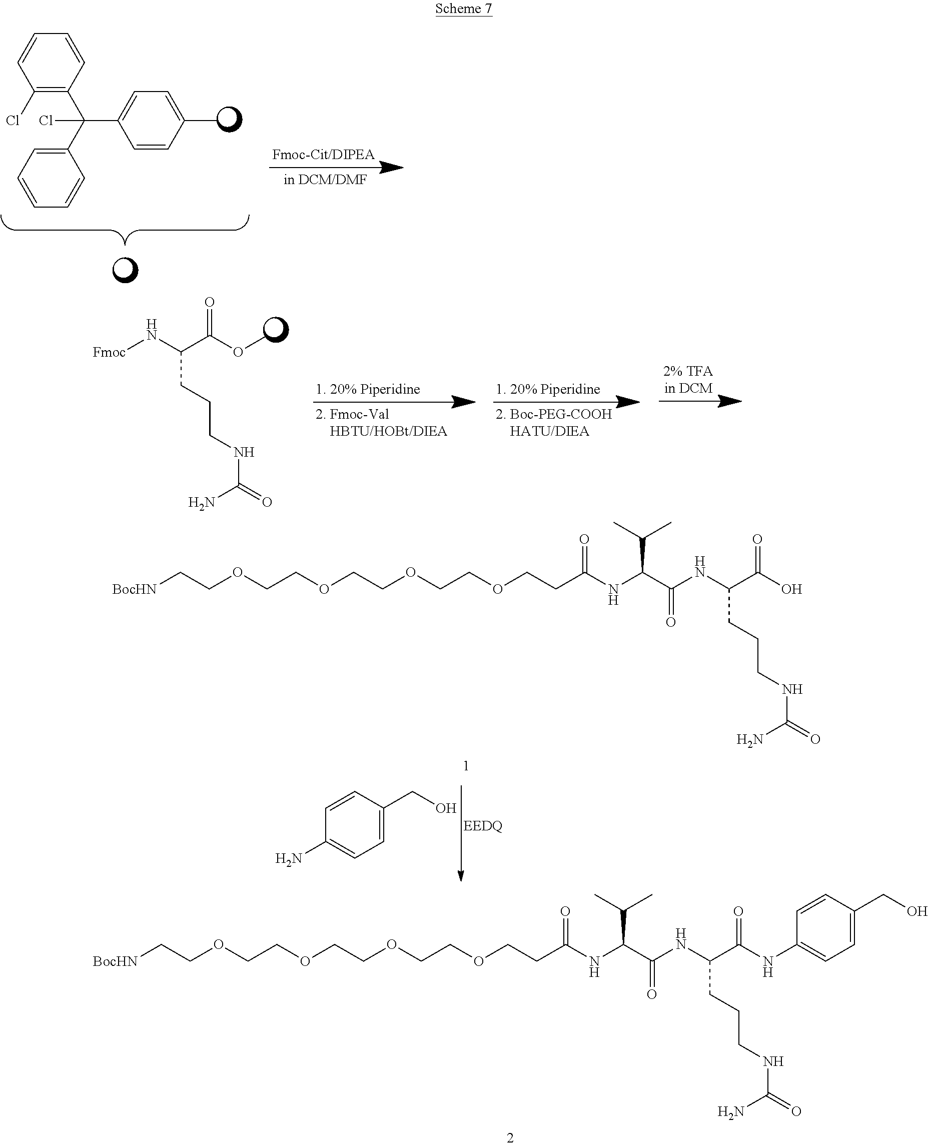

Method 2.

Synthesis on solid phase by attaching Fmoc-Cit-PABA to the 2-chlorotrityl resin followed by coupling with Fmoc-Val-OH and Boc-PEG-COOH using standard protocols (Scheme 8) was also carried out. Product was cleaved with 2% TFA and characterized using analytical HPLC and MS. The target Boc-PEG4-Val-Cit-PABA 2 was obtained with low yields.

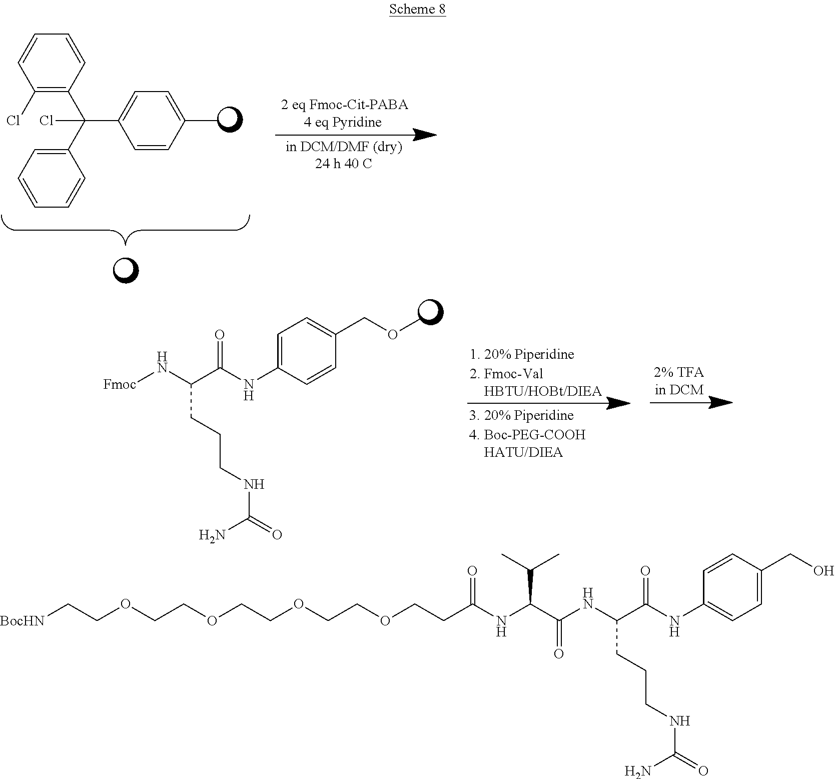

Method 3.

A third method, which was a fully solution-based approach, was also used with good yields (Scheme 9). The NHS ester 1 was prepared from the corresponding carboxylic acid and then coupled with L-valine to give 2. Fmoc L-citrulline was coupled with PABA under literature conditions to give 4 in good yield. Fmoc cleavage, followed by chromatographic purification of one ion gave 5, which was coupled with the valine derivative 3 under conventional conditions to give 2, which was purified by silica gel chromatography. The benzylic hydroxyl group of mining the origin reacted with p-nitrophenylchloroformate to give the active carbonate 6. 1H NMR and LCMS analysis revealed a small amount of a diastereomeric impurity in both 2 and 6, which was not separable by normal phase silica gel chromatography but was eventually removed from 6 by careful preparative RP-HPLC. The origin of this impurity has not been established and could arise from either a) the presence of some of the enantiomer in one of the amino acid starting materials or b) partial epimerisation of one of the amino acids during one of the coupling steps. A possible approach to determining the origin of the diastereoisomerism would be to use the amine-reactive homochiral FLEC reagent (see Camerino et al. 2013 and refs therein) to derivatize valine starting material and/or the citrulline derivative 5 and quantify diastereomeric impurities by conventional HPLC (LC-MS).

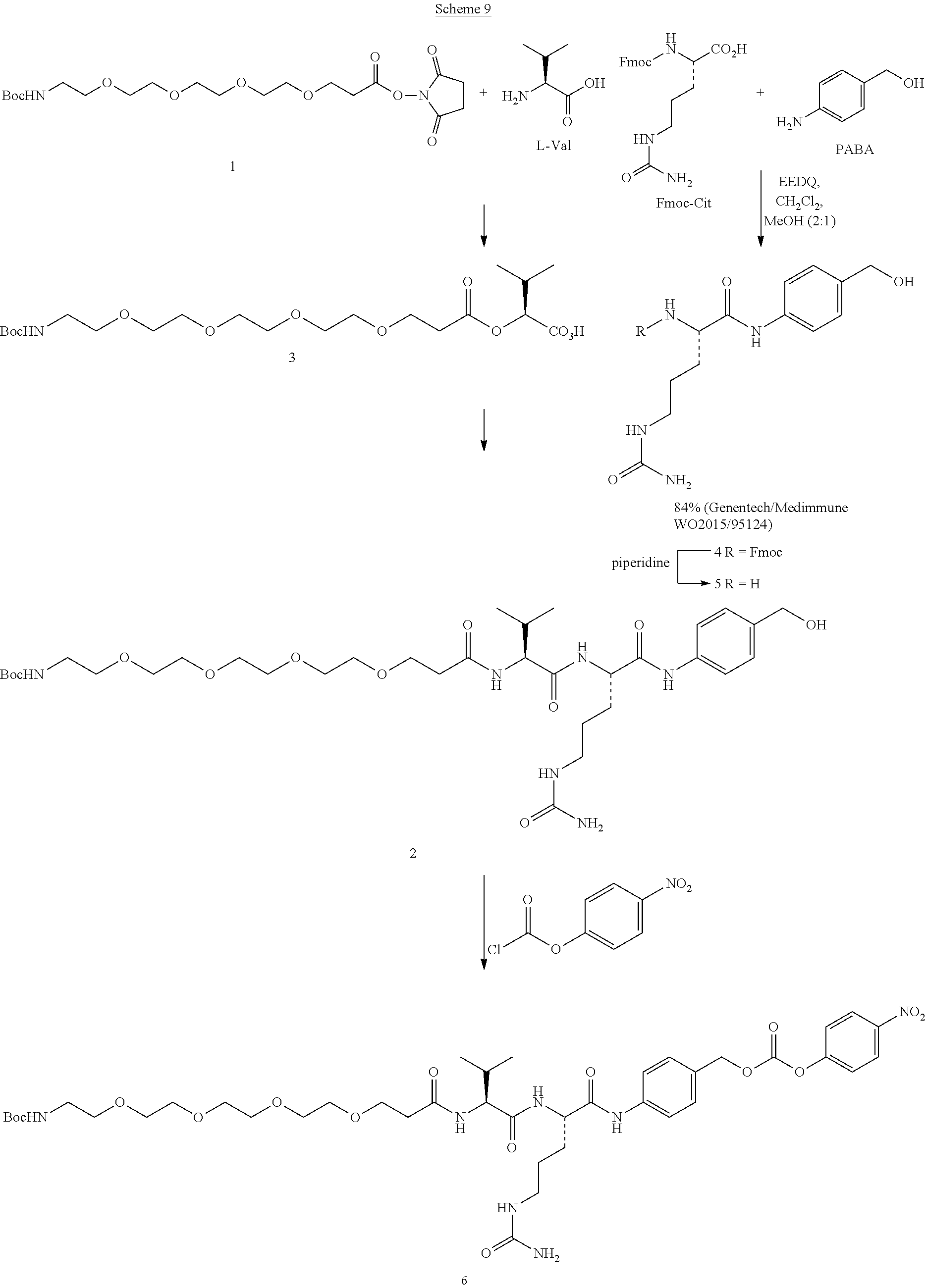

The p-nitrophenylcarbonate 6 was treated with MMAE in the presence of diisopropylethylamine (DIPEA) to give the conjugate 7 (Scheme 10). Conjugate 7 was not purified but was subjected directly to Boc-deprotection then purified by RP-HPLC, to give the amino-terminal PEG-Val-Cit-PABA-MMAE construct 8. This peptide-linker-MMAE construct (8) was obtained in a homogeneous form (isolated purity >95% by HPLC), with MS data consistent with theoretical data. This construct was then used for loading to the copolymer discussed below.

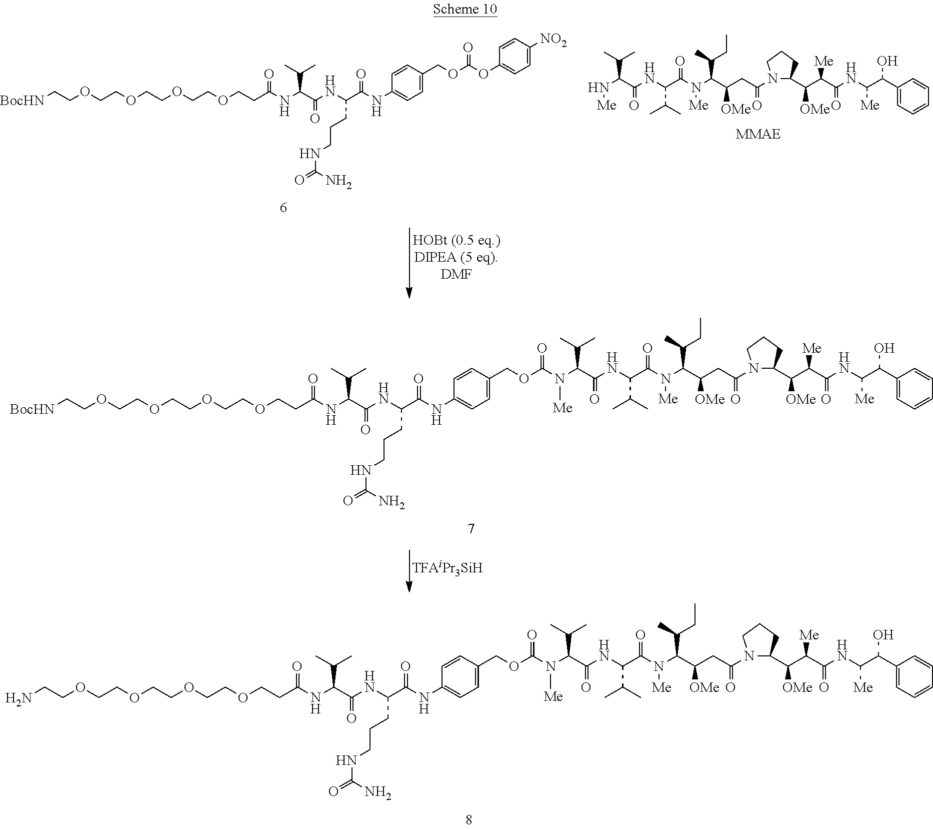

Preparation of Terpolymer-Drug Conjugate

Drug conjugation was done post-polymerisation, after formation of a terpolymer from polymerisation of a monomer composition containing three different monomers, HPMA, NIPAM and a low percentage of a third monomer, N-acryloxysuccinimide (NAS). The optimised polymerisation reaction gave the desired ratio of HPMA to NIPAM (either 1:1 or 1.4:1) with either NAS 10 or NAS 20 included in the backbone (Scheme 11). NAS 20 gave the best combination of low PDI, high MW and importantly the highest number of reactable succinimide groups in the terpolymepolymer. The abbreviation, NAS 10 or NAS 20 refers to the feed ratio (either 10% or 20%) of NAS to RAFT agent.

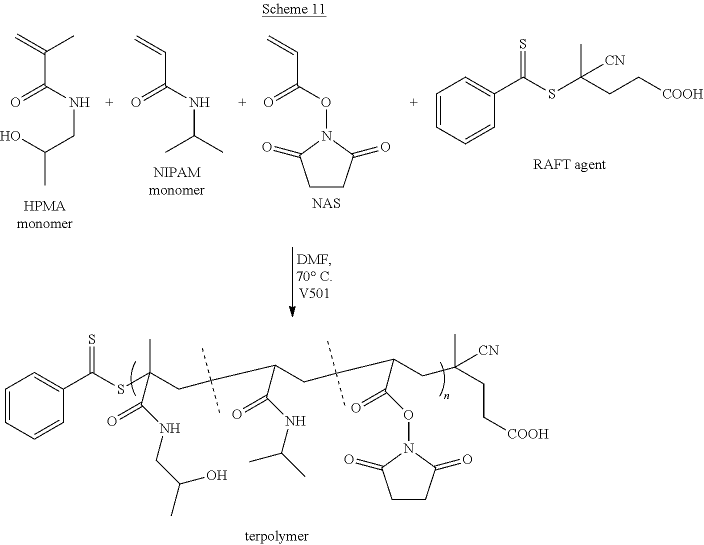

Synthesis of Terpolymer Backbone:

To form the terpolymer backbone, the three monomers, HPMA monomer, NIPAM monomer and NAS monomer (either 10% or 20%), along with 4-cyano-4-(thiobenzoylthio)pentanoic acid (RAFT agent) and 4,4′-azobis(4-cyanovaleric acid) (initiator; V501) were dissolved in DMF. The reaction solution was degassed by nitrogen bubbling for 30 min and then stirring at 70° C. Monomer conversion was monitored by 1H NMR to control the overall polymer molecular weight. After 11 hours, the reaction was stopped by cooling to room temperature. The terpolymer was purified by precipitation in diethyl ether. The molecular weight (MW) of the resulting terpolymer was about 30 kDa.

The terpolymer was loaded with drug (either single drug loading or multiple drug loading) as follows:

Single Drug Loading:

For single drug loading, the drug is attached to an end of the thiol functionalised terpolymer (following RAFT end group removal) in accordance with Scheme 12.

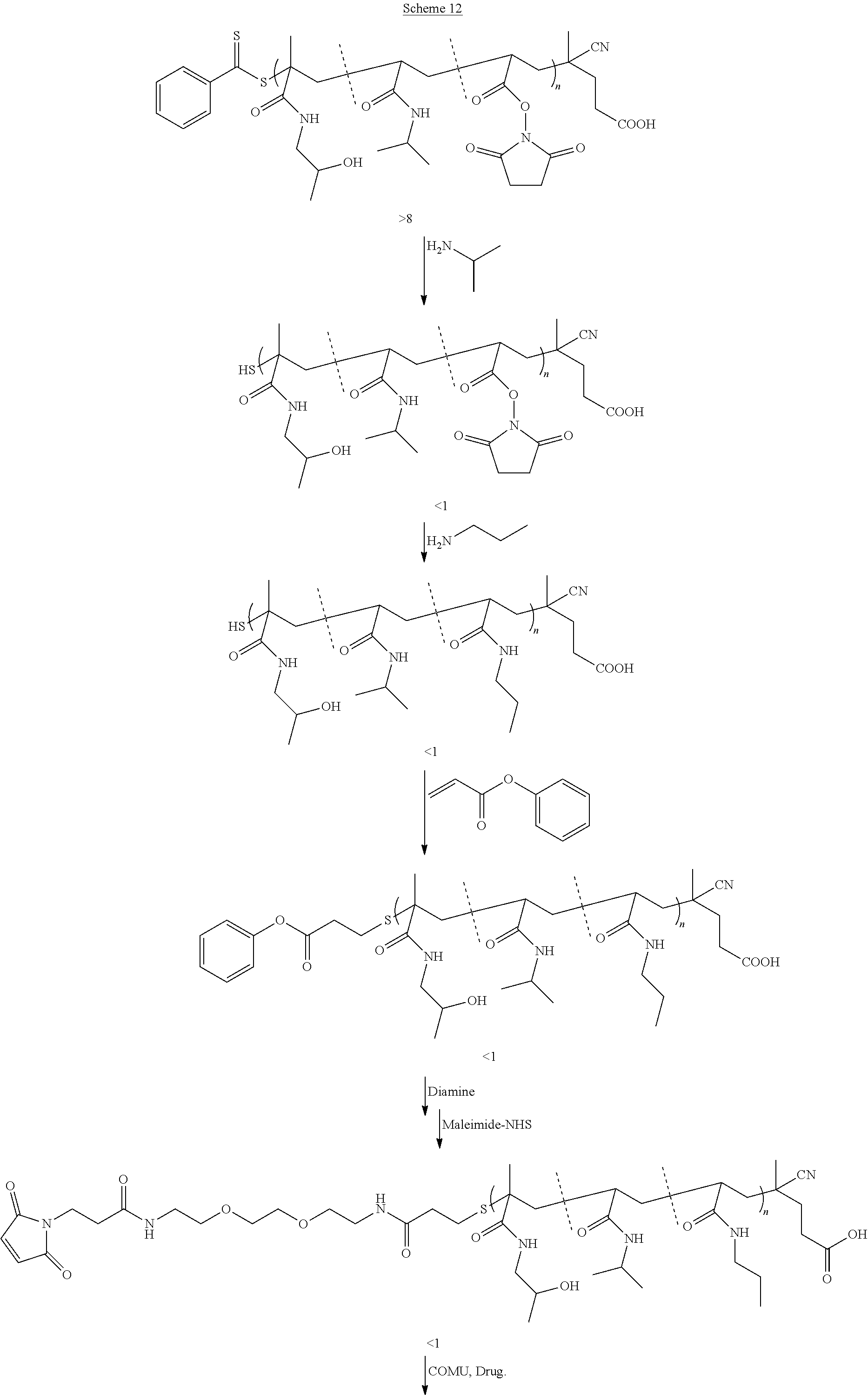

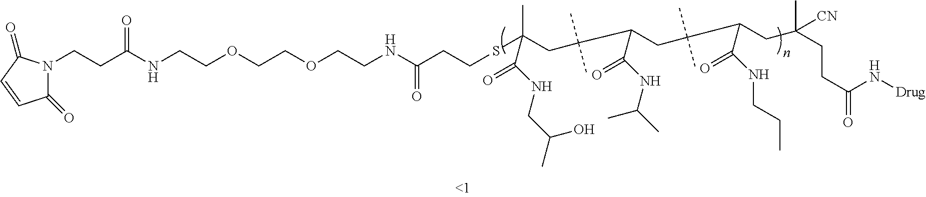

The above maleimide functionalised single drug loaded terpolymer was prepared as follows:

Step 1:

A pHPMA-NiPAM-NAS terpolymer was prepared as described above. The terpolymer was treated with an excess of hexylamine to remove the RAFT end group and provide a terminal thiol (SH) functional group at the end of the terpolymer.

Step 2:

NHS-ester functional groups pendant from the terpolymer (that were introduced using the NAS monomer) were reacted with an excess of propyl amine (20 eq.) in DMF to covert the NHS-ester into non-reactive alkyl amide pendant groups.

Step 3:

The terminal thiol functionality at the end of the terpolymer was reacted with excess phenyl acrylate (10 eq.) in DMF to provide a terminal active ester. The terminal ester group was reacted with excess PEG diamine, leaving a terminal amine at the end of the polymer. The amine terminated terpolymer was then reacted with succinimidyl 3-maleimidopropionate (maleimido-NHS) to install a maleimide group at one end of the polymer for conjugation to an antibody fragment (Fab′). The resulting MAL-functionalised terpolymer was purified by dialysis against water.

Step 4:

The carboxylic acid functional group at the other end of the terpolymer was reacted with the drug-linker-amine in the presence of peptide coupling agents to install the drug at the other end of the polymer to the maleimide, which will be conjugated to the Fab′.

Step 5:

The maleimide functionalised terpolymer from step 3 was then reacted with PEG-Val-Cit-PABA-MMAE (8) as an amino-terminal drug-containing compound to couple the drug MMAE to the terminal carboxylic acid functional group at the other end of the terpolymer. In this step, the maleimide-containing terpolymer (0.012 mmol, 1 eq), PEG-Val-Cit-PABA-MMAE (8) (0.06 mmol, 5 eq) and diisopropylethylamine (0.12 mmol, 10 eq) and 1-cyano-2-ethoxy-2-oxoethylidenaminooxy)dimethylamino-morpholino-carbenium hexafluorophosphate (COMU, 0.0147 mmol, 1.1 eq) were dissolved in 5 mL dimethylformamide (DMF). The mixture was stirred for 5 days at room temperature to conjugate a single drug to the end of the terpolymer.

Multiple Drug Loading:

For multiple drug loading, the drug is attached to pendant to the terpolymer (following RAFT end group removal) in accordance with Scheme 13.

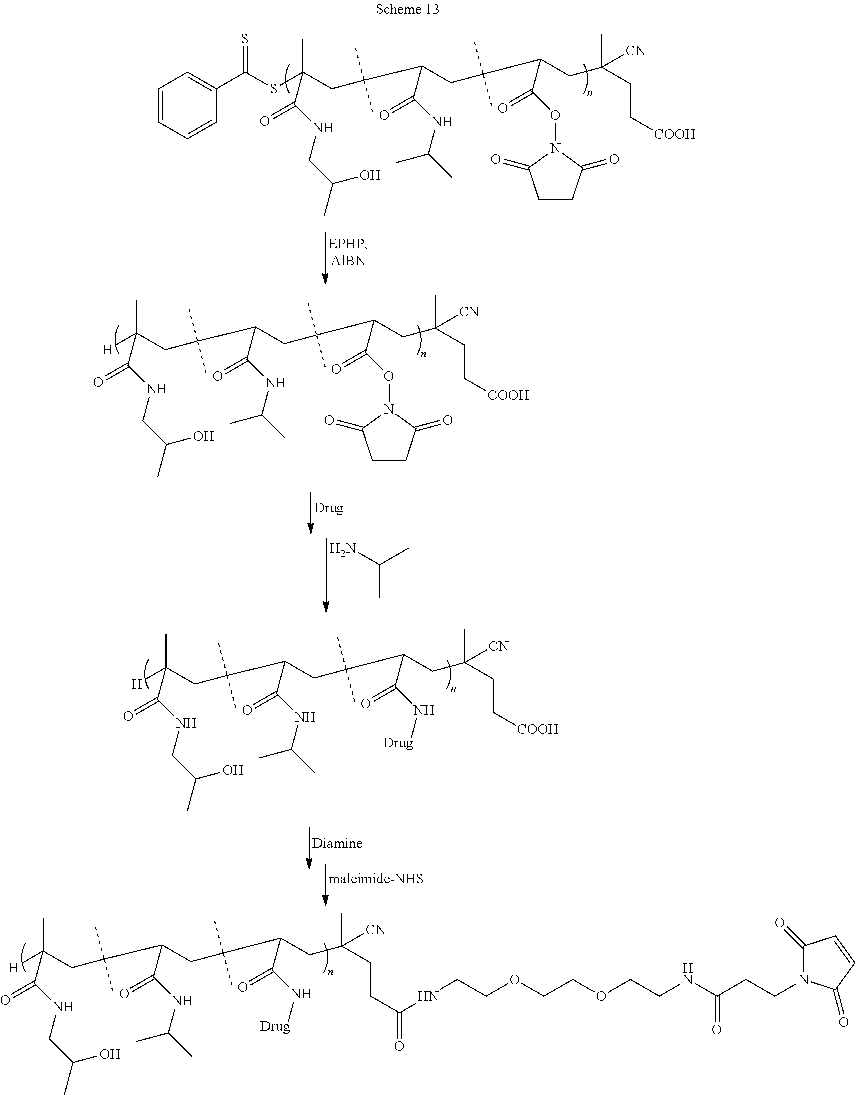

Step 1:

A pHPMA-NiPAM-NAS terpolymer (0.0243 mmol, 1 eq), azobisisobutyronitrile (AIBN, 0.0365 mmol, 1.5 eq), and N-ethylpiperidine hypophosphite (EPHP, 1.22 mmol, 50 eq) were dissolved in 50 mL dimethylacetamide. The mixture was degassed, by bubbling through N₂ for 40 mins, and then heated to 80° C. for 16 h. The resulting terpolymer was purified by precipitation from methanol into diethyl ether three times.

Step 2:

The terpolymer from step 1 (0.012 mmol, 1 eq), amino terminal PEG-Val-Cit-PABA-MMAE (8) as an amino-terminal drug-containing compound (0.06 mmol, 5 eq) and diisopropylethylamine (0.12 mmol, 10 eq) were dissolved in 5 mL dimethylformamide (DMF). The mixture was stirred for 5 days at room temperature. Isopropylamine (12 mmol, 100 eq) was added to the stirred solution and left overnight. Propylamine (12 mmol, 100 eq) was then added to the stirred solution and left overnight. These last two steps were to ensure the NHS ester from the NAS in the terpolymer was capped with a small molecule amine. The product was purified by dialysis against water.

Step 3:

At room temperature, (1-cyano-2-ethoxy-2-oxoethylidenaminooxy)dimethylamino-morpholino-carbenium hexafluorophosphate (COMU, 0.0147 mmol, 1.1 eq) and diisopropylethylamine (0.0486 mmol, 4 eq) were added to the product from step 2 in DMF (5 mL) with stirring. After 2 mins, PEG₂-diamine (0.243 mmol, 20 eq) was added. After 4 hours, diisopropylethylamine (0.728 mmol, 60 eq) and succinimidyl 3-maleimidopropionate (maleimide-NHS) (0.728 mmol, 60 eq) were added with continued stirring. The solution was stirred at room temperature for another 2 hours, the product was purified by dialysis against water.

The final ratio of NIPAM monomer to HPMA monomer is about 2 to 3 (NIPAM DP 103 to HPMA DP 147). The NAS monomer (containing NHS ester) was DP 20.

For both the single-drug and multi-drug loaded terpolymers prepared above, the maleimide-containing linker was installed at the end of the terpolymer using similar chemistry to that described previously. About 400 mg of each of the single drug loaded terpolymer and multiple drug loaded terpolymer were prepared for bioconjugation to the Fab′.

Conjugation of Protein Fab′ to a Terminal End of Drug Loaded Polymer

To Fab′2 (173 mg, 1.7 mmol) in PBS reduced with 1.5 equivalents of TCEP for 2 h at 25° C. was added 1.5 equivalents of maleimide-containing RAFT terpolymer with either single or multiple drug loading, prepared as above. The mixture was allowed to react for 1 h at 4° C. with mixing. The mixture was diluted 10-fold with 10 mM MES pH 6 and applied to two 5 mL HiTrap SP HP columns (GE Healthcare) connected in series. The unbound material was washed and the protein eluted with a linear gradient from 0-1 M NaCl in the same buffer. The early eluting material was pooled and subjected to gel filtration on a Superdex S 200 2660 column. The pooled material was concentrated and found to be greater than 95% pure as judged by the gel filtration profile (Superdex S200 1030 column).

For tumour reduction animal studies it was important to purify the conjugates to remove unconjugated drug and/or Fab′. Isolation was by ion exchange followed by gel filtration. Table 7 summarises the various conjugates made for the animal studies.

TABLE 7

| | Conjugated to | | | |
|---|---|---|---|---|
| Group | one end of the | NAS modification post- | | Number |
| and dose | polymer | polymerisation | Final conjugate | of drugs |
| 1 high | n/a | n/a | Positive Control Antibody | n/a |
| 2 high | n/a | n/a | Negative Control Antibody | n/a |
| 3 high | | H2N-PEG-ValCitPABA- | Terpolymer(MMAE)x | Multi drug, |
| | | MMAE | | NO Fab |
| 4 high | | H2N-PEG-ValCitPABA- | Terpolymer(MMAE)x-Fab | Multi |
| | | MMAE | | |
| 5 low | n/a | n/a | Positive Control Antibody | n/a |
| 6 high | H2N-PEG- | isopropylamine | MMAE-terpolymer-Fab | 1 |
| | ValCitPABA- | | | |
| | MMAE | | | |
| 7 low | | H2N-PEG-ValCitPABA- | Terpolymer(MMAE)x-Fab | Multi |
| | | MMAE | | |
| 8 high | | H2N-PEG-ValCitPABA- | Fab-PEG₂₄-MMAE | 1 drug, |
| | | MMAE | | NO |
| | | (on Fab′ via NHS-PEG₂₄- | | terpolymer |
| | | MAL linker) | | |

In Table 7:
Groups 1, 2 and 5 are positive and negative antibody controls.
Group 3 represents a terpolymer-drug conjugate with multiple pendant drugs and no Fab′ fragment (binding moiety)
Groups 4, 6 and 7 represent drug-terpolymer-Fab′ conjugates of the invention with either single drug or multiple drug loading, which were assessed at different doses. The drug was pendant in terpolymer(MMAEA)x-Fab and at the end of the polymer in MMAE-terpolymer-Fab.
Group 8 represents a drug-antibody conjugate with no terpolymer.

Tumour Reduction Studies

Drug-Loaded Terpolymer-Antibody Conjugates.

Drug-polymer-Fab′ conjugates with single or multiple drug loading were prepared in accordance with the procedure described above and assessed in a tumour burden animal study.

Efficacy Study:

Seventy-five female FoxN1 nu mice bearing subcutaneously inoculated A431 epidermoid tumours were randomly assigned into eight groups 10 days post-inoculation (Study Day 0), when mean tumour volume was approximately 119 mm3 (variability of 2.7%). Animals were assigned to eight different groups. Animals in each group received intravenous tail vein treatment with one of the control antibodies or a test antibody conjugate. Treatments were administered on Study Days 0, 3, 6, 9 and 12. The study was terminated on Study Day 43 for animals that were not euthanised early due to ethical limits. Upon termination, the tumour was excised from all animals and weighed.

The study groups were as follows:
Group 1: Positive Control Antibody, mAb 528; 20 mg/kg in 11.21 mL/kg (n=10);
Group 2: Negative Control Antibody (mAb control; 20 mg/kg in 10.14 mL/kg (n=10);
Group 3: Polymer-drug4, RAFT-MMAE-DAR4 (14.4 mg/kg in 14.4 mL/kg (n=10);
Group 4: (Terpolymer) Fab-terpolymer-drug4, Fab-RAFT-MMAE-DAR4 (33.4 mg/kg in 15.61 mL/kg) (n=9);
Group 5: Positive Control Antibody, mAb 528; 10 mg/kg in 5.61 mL/kg (n=10);
Group 6: (Terpolymer) Fab-terpolymer-drug1, Fab-RAFT-MMAE-DAR1 (32 mg/kg in 13.41 mL/kg) (n=9);
Group 7: (Terpolymer) Fab-terpolymer-drug4, Fab-RAFT-MMAE-DAR4 (9.3 mg/kg in 4.35 mL/kg (n=10);
Group 8: Fab-drug1, Fab-PEG₂₄-MMAE (20 mg/kg in 19.86 mL/kg) (n=7).

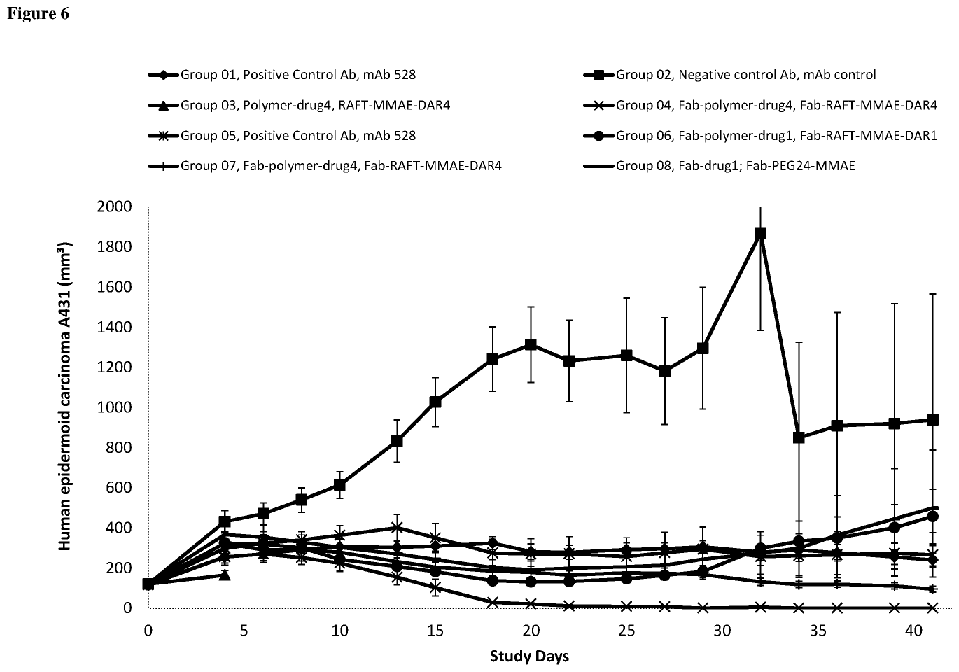

The results are shown in FIG. 6.

As seen in FIG. 6, treatment with Positive Control; mAb 528, (Groups 1 & 5), Fab-polymer-drug1: Fab-RAFT-MMAE-DAR1 (Group 6—single drug loading), Fab-polymer-drug4: Fab-RAFT-MMAE-DAR4 (Group 7—multiple drug loading) and Fab-drug1; Fab-PEG₂₄-MMAE, Group 8) resulted in significant inhibition of tumour growth on Study Day 20 compared with Negative Control Ab; mAb control (Group 2).

FIG. 6 also shows there was no difference in tumour growth inhibition in groups treated with Positive Control (Group 5), Fab-polymer-drug1, Fab-RAFT-MMAE-DAR1 (Group 6), Fab-polymer-drug4, Fab-RAFT-MMAE-DAR4 (Group 7) and Fab-drug1; Fab-PEG₂₄-MMAE (Group 8) compared with Positive Control Ab, Group 1).

It is to be understood that various other modifications and/or alterations may be made without departing from the spirit of the present invention as outlined herein.

1. A biocompatible, hydrophilic polymer conjugate comprising:
a linear, aliphatic, statistical copolymer backbone having two ends and being derived from at least three different ethylenically unsaturated monomers;
a binding moiety conjugated to an end of the copolymer backbone; and
at least one agent conjugated to the copolymer backbone.

2. The conjugate according to claim 1, wherein the different monomers each have different ethylenically unsaturated groups.

3. The conjugate according to claim 2, wherein the different monomers belong to classes of monomer selected from acrylate, methacrylate, acrylamido, methacrylamido and vinyl ester.

4. The conjugate according to claim 1, wherein the copolymer backbone is a terpolymer derived from three different ethylenically unsaturated monomers, wherein each monomer has a different ethylenically unsaturated group.

5. The conjugate according to claim 1, wherein the copolymer backbone is derived from:
(a) a first monomer selected from N-(2-hydroxypropyl)methacrylamide and N-(2-hydroxypropyl)acrylamide;
(b) a second monomer selected from the group consisting of 2-hydroxyethyl acrylate, 2-methoxyethyl acrylate, 2-(diethylene glycol) ethyl acrylate, poly(ethylene glycol) acrylate, poly(ethylene glycol) methacrylate, poly(ethylene glycol) methyl ether acrylate, poly(ethylene glycol) methyl ether methacrylate, N-acryloylamido-ethoxyethanol, N,N-dimethylacrylamide, N,N-diethylacrylamide, N-(2-hydroxyethyl) acrylamide, N-(2-hydroxyethyl) methacrylamide, N-[Tris(hydroxymethyl)methyl]acrylamide, acrylamide, N-acryloylmorpholine, N-propyl acrylamide, N-isopropyl acrylamide, methacrylamide, di(ethylene glycol) methyl ether methacrylate, 2-hydroxyethyl methacrylate, 2-(dimethylamino) ethyl acrylate, 2-(diethylamino) ethyl acrylate, 3-(dimethylamino) propyl acrylate, (3-acrylamidopropyl) trimethylammonium chloride, 2-aminoethyl methacrylate hydrochloride, [3-(methacryloylamino)propyl]trimethylammonium chloride, 2-carboxyethyl acrylate, acrylic acid, N-carboxyethyl acrylamide, 2-acrylamido-2-methyl-1-propane sodium sulfonate, 3-sulfopropyl methacrylate potassium salt, methacrylic acid, 3[[2-dimethylammonio]propionate, [2-(methacryloyloxy)ethyl]dimethyl-(3-sulfopropyl)ammonium hydroxide, and 2-methacryloyloxyethyl phosphorylcholine, 3-dimethyl-ammonio]propionate, 2-acryloyloxyethyl phosphorylcholine, [2-(acryloyloxy)ethyl]dimethyl-(3-sulfopropyl) ammonium hydroxide, N-(2-propynyl)-acrylamide, N-(3-azidopropyl)-acrylamide, N-(3-azidopropyl)-methacrylamide, and vinyl ester; and
(c) a third monomer selected from an acryloyl or methacryloyl monomer comprising a functional group capable of reacting with an agent-containing molecule, and an acryloyl or methacryloyl monomer comprising an agent conjugated thereto.

6. The conjugate according to claim 1, wherein the agent is conjugated to an end of the copolymer backbone, with the proviso that the agent and binding moiety are conjugated to different ends.

7. The conjugate according to claim 1, wherein the agent is conjugated to and pendant from the copolymer backbone.

8. The conjugate according to claim 1, wherein the copolymer backbone has a molecular weight of no more than about 40 kDa.

9. The conjugate according to claim 1, wherein the copolymer backbone has a polydispersity of no more than about 1.5.

10. The conjugate according to claim 1, wherein the binding moiety is selected from the group consisting of an antibody, an antibody fragment, and an antigen binding fragment.

11. The conjugate according to claim 1, wherein the binding moiety is a Fab′ fragment.

12. The conjugate according to claim 1, wherein the binding moiety is conjugated to the copolymer backbone via a linker comprising a moiety of formula (I): 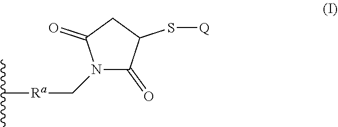
where:
Q represents the binding moiety;
R^{a} represents the remainder of the linker; and [symbol]
represents a site of attachment to an end of the copolymer backbone.

13. The conjugate according to claim 1, comprising a diagnostic agent or a therapeutic agent conjugated to the copolymer backbone.

14. The conjugate according to claim 1, comprising a therapeutic agent that is conjugated to the copolymer backbone via biodegradable linker.

15. The conjugate according to claim 14, wherein the biodegradable linker comprises a moiety selected from the group consisting of valine-citrulline-para-aminobenzoic acid (Val-Cit-PABA), valine-alanine (Val-Ala), and phenylalanine-lysine (Phe-Lys).

16. A process for preparing a biocompatible, hydrophilic polymer conjugate, the process comprising the steps of:
(a) polymerising a monomer composition comprising at least three different ethylenically unsaturated monomers under conditions of free radical polymerisation to form a linear, aliphatic, statistical copolymer backbone having two ends, a first functional group for conjugating a binding moiety at a first end of the copolymer backbone, and a second functional group for conjugating an agent at a position selected from the second end of the copolymer backbone and pendant from the copolymer backbone;
(b) covalently reacting the first functional group with a binding moiety-containing molecule to conjugate the binding moiety to the first end of the copolymer backbone; and
(c) covalently reacting the second functional group with an agent-containing molecule to conjugate the agent to the copolymer backbone at a position selected from the second end of the copolymer backbone and pendant from the copolymer backbone.

17. The process according to claim 16, wherein the different monomers in the monomer composition have different ethylenically unsaturated groups.

18. The process according to claim 17, wherein the different monomers belong to classes of monomer selected from acrylate, methacrylate, acrylamido, methacrylamido and vinyl ester.

19. The process according to claim 16, wherein the monomer composition is polymerised under conditions of living free radical polymerisation.

20. The process according to claim 16, wherein the monomer composition comprises:
(a) a first monomer selected from N-(2-hydroxypropyl)methacrylamide and N-(2-hydroxypropyl)acrylamide;
(b) a second monomer selected from the group consisting of 2-hydroxyethyl acrylate, 2-methoxyethyl acrylate, 2-(diethylene glycol) ethyl acrylate, poly(ethylene glycol) acrylate, poly(ethylene glycol) methacrylate, poly(ethylene glycol) methyl ether acrylate, poly(ethylene glycol) methyl ether methacrylate, N-acryloylamido-ethoxyethanol, N,N-dimethylacrylamide, N,N-diethylacrylamide, N-(2-hydroxyethyl) acrylamide, N-(2-hydroxyethyl) methacrylamide, N [Tris(hydroxymethyl)methyl]acrylamide, acrylamide, N-acryloylmorpholine, N propyl acrylamide, N isopropyl acrylamide, methacrylamide, di(ethylene glycol) methyl ether methacrylate, 2 hydroxyethyl methacrylate, 2-(dimethylamino) ethyl acrylate, 2-(diethylamino) ethyl acrylate, 3-(dimethylamino) propyl acrylate, (3 acrylamidopropyl) trimethylammonium chloride, 2-aminoethyl methacrylate hydrochloride, [3-(methacryloylamino)propyl]trimethylammonium chloride, 2 carboxyethyl acrylate, acrylic acid, N-carboxyethyl acrylamide, 2-acrylamido-2-methyl-1-propane sodium sulfonate, 3-sulfopropyl methacrylate potassium salt, methacrylic acid, 3[[2-dimethylammonio]propionate, [2-(methacryloyloxy)ethyl]dimethyl-(3-sulfopropyl)ammonium hydroxide, and 2-methacryloyloxyethyl phosphorylcholine, 3[[2-dimethyl-ammonio]propionate, 2-acryloyloxyethyl phosphorylcholine, [2-(acryloyloxy)ethyl]dimethyl-(3-sulfopropyl) ammonium hydroxide, N-(2-propynyl)-acrylamide, and N-(3-azidopropyl)-acrylamide; and
(c) a third monomer which is a hydrophilic acryloyl or methacryloyl monomer comprising a functional group that is capable of reacting with an agent-containing molecule for conjugation of the agent to the copolymer backbone.

21. The process according to claim 20, wherein the third monomer is acryloyloxysuccinimide.

22. The process according to claim 16, wherein the binding moiety-containing molecule comprises an antibody, an antibody fragment, and an antigen binding fragment.

23. A process for preparing a biocompatible, hydrophilic polymer conjugate, the process comprising the steps of:
(a) polymerising a monomer composition comprising at least two different ethylenically unsaturated monomers and an ethylenically unsaturated monomer-agent conjugate under conditions of free radical polymerisation to thereby form a linear, aliphatic statistical copolymer backbone having a pendant agent and a terminal functional group at one or both ends of the copolymer backbone;
(b) covalently reacting a binding moiety-containing molecule with a first terminal functional group at a first end of the copolymer backbone to conjugate the binding moiety to the first end; and
optionally (c) covalently reacting an agent-containing molecule with a second terminal functional group at a second end of the copolymer backbone to conjugate the agent to the second end.

24. The process according to claim 23, wherein the ethylenically unsaturated monomers and the monomer-agent conjugate have different ethylenically unsaturated groups.

25. The process according to claim 23, wherein the monomer composition is polymerised under conditions of living free radical polymerisation, preferably reversible-addition-fragmentation-chain transfer (RAFT) polymerisation.

26. The process according to claim 23, wherein the monomer composition comprises:
(a) a first monomer selected from N-(2-hydroxypropyl)methacrylamide and N-(2-hydroxypropyl)acrylamide;
(b) a second monomer selected from the group consisting of 2-hydroxyethyl acrylate, 2-methoxyethyl acrylate, 2-(diethylene glycol) ethyl acrylate, poly(ethylene glycol) acrylate, poly(ethylene glycol) methacrylate, poly(ethylene glycol) methyl ether acrylate, poly(ethylene glycol) methyl ether methacrylate, N-acryloylamido-ethoxyethanol, N,N-dimethylacrylamide, N,N-diethylacrylamide, N-(2-hydroxyethyl) acrylamide, N-(2-hydroxyethyl) methacrylamide, N [Tris(hydroxymethyl)methyl]acrylamide, acrylamide, N-acryloylmorpholine, N propyl acrylamide, N isopropyl acrylamide, methacrylamide, di(ethylene glycol) methyl ether methacrylate, 2 hydroxyethyl methacrylate, 2-(dimethylamino) ethyl acrylate, 2-(diethylamino) ethyl acrylate, 3-(dimethylamino) propyl acrylate, (3 acrylamidopropyl) trimethylammonium chloride, 2-aminoethyl methacrylate hydrochloride, [3-(methacryloylamino)propyl]trimethylammonium chloride, 2 carboxyethyl acrylate, acrylic acid, N-carboxyethyl acrylamide, 2-acrylamido-2-methyl-1-propane sodium sulfonate, 3-sulfopropyl methacrylate potassium salt, methacrylic acid, 3-dimethylammonio]propionate, [2-(methacryloyloxy)ethyl]dimethyl-(3-sulfopropyl)ammonium hydroxide, and 2-methacryloyloxyethyl phosphorylcholine, 3-dimethyl-ammonio]propionate, 2-acryloyloxyethyl phosphorylcholine, [2-(acryloyloxy)ethyl]dimethyl-(3-sulfopropyl) ammonium hydroxide, N-(2-propynyl)-acrylamide, and N-(3-azidopropyl)-acrylamide; and
(c) a third monomer which is a monomer-agent conjugate of formula (III): 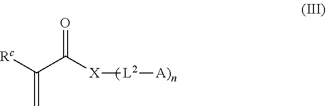
where:
R^{c} is H or CH₃;
X is selected from O or N;
L² represents a linking moiety;
A represents an agent; and
n represents the number of (-L²-A) groups attached to X and is 1 or 2.

27. The process according to claim 26, wherein formula (III), A is a therapeutic agent and L² is a biodegradable linking moiety.

28. The process according to claim 23, wherein the binding moiety-containing molecule comprises an antibody, an antibody fragment, and an antigen binding fragment.

29. A method of alleviating, treating or preventing a disease or disorder in a subject comprising the step of administering to the subject; an effective amount of the polymer conjugate of claim 1.

30. A method of delivering an agent to a target cellular or tissue site in a subject, the method comprising the step of administering to the subject; an effective amount of the polymer conjugate of claim 1.
